SEQUENCE LISTING (1 sequence)

SEQ ID NO: 1 - protein, 20 aa, synthetic construct
RITFGGPSDSTGSNQNGERS

(19) United States
(12) Patent Application Publication
(10) Pub. No.: US 2024/0361215 A1
(43) Pub. Date: Oct. 31, 2024

(54) FLUID SAMPLE CONCENTRATOR

(71) Applicant: University of Washington, Seattle, WA (US)

(72) Inventors: Jriuan Lai, Seattle, WA (US); Sheng-You Chen, Seattle, WA (US); Ruby Lunde, Seattle, WA (US); Yuan-Che Wu, Seattle, WA (US)

(73) Assignee: University of Washington, Seattle, WA (US)

(21) Appl. No.: 18/682,859

(22) PCT Filed: Aug. 8, 2022
(86) PCT No.: PCT/US2022/074667

§ 371 Date: Feb. 9, 2024

Related U.S. Application Data
(60) Provisional application No. 63/331,446, filed on Apr. 15, 2022.
(60) Provisional application No. 63/231,044, filed on Aug. 9, 2021.

(51) Int. Cl. G01N 1/40; G01N 33/543; G01N 33/569; G01N 33/76

(52) U.S. Cl. CPC G01N 1/40; G01N 33/54388; G01N 33/56983; G01N 33/76; G01N 2333/165; G01N 2333/59; G01N 2469/10

(57) ABSTRACT

Techniques for concentrating analytes in fluid samples are described. An example method performed by a concentrator includes receiving a fluid sample; concentrating an analyte in the fluid sample by extracting water from the fluid sample; and based on concentrating the analyte in the fluid sample, outputting the fluid sample. The analyte is concentrated by extracting the water through a membrane and into a solution including at least one solute at a concentration that is greater than a concentration of at least one solute in the fluid sample, diameters of pores extending through the membrane being shorter than a diameter of the analyte and shorter than a diameter of the at least one solute.

### CROSS-REFERENCE TO RELATED APPLICATIONS

This application is a U.S. National Phase Application based on International Patent Application No. PCT/US2022/074667, filed on Aug. 8, 2022, which claims the priority of U.S. Provisional App. No. 63/231,044, filed on Aug. 9, 2021 as well as U.S. Provisional App. No. 63/331,446, filed on Apr. 15, 2022, each of which is incorporated by reference herein in its entirety.

### STATEMENT REGARDING FEDERALLY SPONSORED RESEARCH OR DEVELOPMENT

This invention was made with government support under Grant No. AI140460, awarded by the National Institutes of Health. The government has certain rights in the invention.

### REFERENCE TO SEQUENCE LISTING

The present application is being filed along with a Sequence Listing in electronic format. The Sequence Listing is provided as a file entitled W149-0031PCT.xml created on Aug. 8, 2022, which is 2 kilobytes in size. The information in the electronic format of the Sequence Listing is incorporated herein by reference in its entirety.

### TECHNICAL FIELD

This application relates to techniques for increasing the concentration of a target in a fluid sample by drawing a solvent out of the fluid sample, across a membrane, and into a concentrated solution.

### BACKGROUND

The presence of an analyte in a fluid biological sample can be used to evaluate the health of the subject from which the sample is obtained. For example, a nasopharyngeal swab can be used to obtain a sample of mucus from inside of a patient's nose. The presence of a virus in the mucus can be indicative of whether the patient has a viral illness. Diagnostic assays can be used to determine whether a particular analyte-of-interest is present in a fluid sample. Lateral flow assays (LFAs) can be particularly helpful for identifying the presence of an analyte in low-resource settings, due to their relatively low cost. However, LFAs may not be sufficiently sensitive to detect many analytes-of-interest in real-world fluid samples.

### SUMMARY

The present disclosure describes systems, devices, and techniques for concentrating a target (e.g., analyte) in a fluid sample. In some cases, the target cannot be sufficiently detected by a diagnostic assay in the fluid sample as-collected. By concentrating the target, the diagnostic assay is enabled to detect the presence of the target in the fluid sample.

In various implementations, a device includes a solution and a semi-permeable membrane. At least one first solute is present in the solution at a higher concentration than at least one second solute in a fluid sample. When the fluid sample is disposed on a side of the membrane, and the solution is disposed on another side of the membrane, a solvent spontaneously flows from the fluid sample into the solution due to osmotic pressure. The movement of the solvent out of the fluid sample concentrates the target in the fluid sample.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example system for detecting the presence of an analyte in a fluid sample.

FIG. 2 is a diagram illustrating an example flow of materials and components for concentrating a fluid sample.

FIG. 3 illustrates an example environment for concentrating a target in a fluid sample.

FIGS. 4A to 4C illustrate cross-sections of various concentrators in accordance with implementations of the present disclosure.

FIG. 5 illustrates an example process for concentrating a fluid sample.

FIG. 6 illustrates an example process for detecting an analyte in a fluid sample.

FIG. 7 illustrates an example of a device for performing one or more functions described herein.

FIGS. 8A to 8C show an example device components, dimension, and assembly workflow.

FIG. 9 illustrates a process of osmosis applied in an example urine specimen processor.

FIGS. 10A to 10C illustrate water removal rates measured in the first experimental example.

FIGS. 11A to 11H illustrate the design and workflow associated with an example osmotic specimen processing device.

FIGS. 12A and 12B illustrate example results associated with detecting hCG hormone and an example LFA test.

FIGS. 13A and 13B illustrate example results associated with detecting SARS-CoV-2 nucleocapsid (N) protein and an example LFA test.

FIG. 14A illustrates a chart showing a calibration using standards, 0, 0.08, 0.4, 2, 10, and 40 ug/mL SARS-CoV-2 N protein. FIG. 14B illustrates specimens' SARS-CoV-2 N protein concentrations before and after osmosis.

FIGS. 15A and 15B illustrate Orbitrap mass spectrometry intensity vs. retention time chromatograms of an example peptide sequence, the peak near 26 minutes.

FIG. 16 illustrates the example device utilized in an experimental example.

FIG. 17 illustrates the workflow of an example device.

FIG. 18 illustrates lateral flow assay results for 0, 0.005, 0.01, 0.02, 0.04, 0.08, and 2 μg/mL hCG solutions.

FIG. 19 illustrates results of examples of 0 μg/mL, 0.02 μg/mL (unprocessed), device-processed, and 2 μg/mL hCG LFAs.

FIG. 20 illustrates hCG LFA test band intensity vs. log hCG concentration plot of 0 ug/mL, 0.02 ug/mL (unprocessed), 2 ug/mL, and device-processed data plotted along a calibration trendline.

FIG. 21 illustrates test band intensity vs. hCG concentration plot of 0 μg/mL, 0.02 μg/mL (unprocessed), 2 μg/mL hCG, and processed hCG samples.

FIG. 22 illustrates an example of water removed vs. PEG molecular weight (prepared at maximum concentrations).

FIGS. 23A to 23D illustrate an example device for concentrating and collecting biomolecules via osmosis.

FIG. 24 illustrates an example concentrator housing.

FIGS. 25A and 25B illustrate an exploded view of an example concentrator.

FIG. 26 illustrates an example of the interior operation of an example concentrator.

FIG. 27 illustrates an example of the exterior operation of an example concentrator.

FIG. 28 illustrates example dimensions of an example concentrator.

FIGS. 29A and 29B illustrate diagrams of an example concentrator.

### DETAILED DESCRIPTION

Various implementations described herein relate to techniques for concentrating analytes in a fluid sample. The fluid sample, for instance, includes urine, blood, saliva, or another type of biological specimen. An example concentrator includes a highly concentrated solution separated from the fluid sample by a porous membrane. The highly concentrated solution, for example, includes a first concentration of at least one first solute (e.g., polymers, micelles, detergents, etc.), whereas the fluid sample includes a second concentration of at least one second solute. The first concentration is greater than the second concentration. Accordingly, a solvent (e.g., water) is spontaneously drawn into the highly concentrated solution through the porous membrane due to osmotic pressure.

The concentrated fluid sample may be tested for an analyte. In some cases, an assay (e.g., a lateral flow test) configured to detect the analyte has a limit of detection that is greater than the concentration of the analyte in the fluid sample before it is concentrated. However, the concentration of the analyte in the concentrated fluid sample is greater than the limit of detection of the assay. Accordingly, various implementations described herein can be used to enable relatively insensitive diagnostic assays to detect trace amounts of an analyte in a fluid sample.

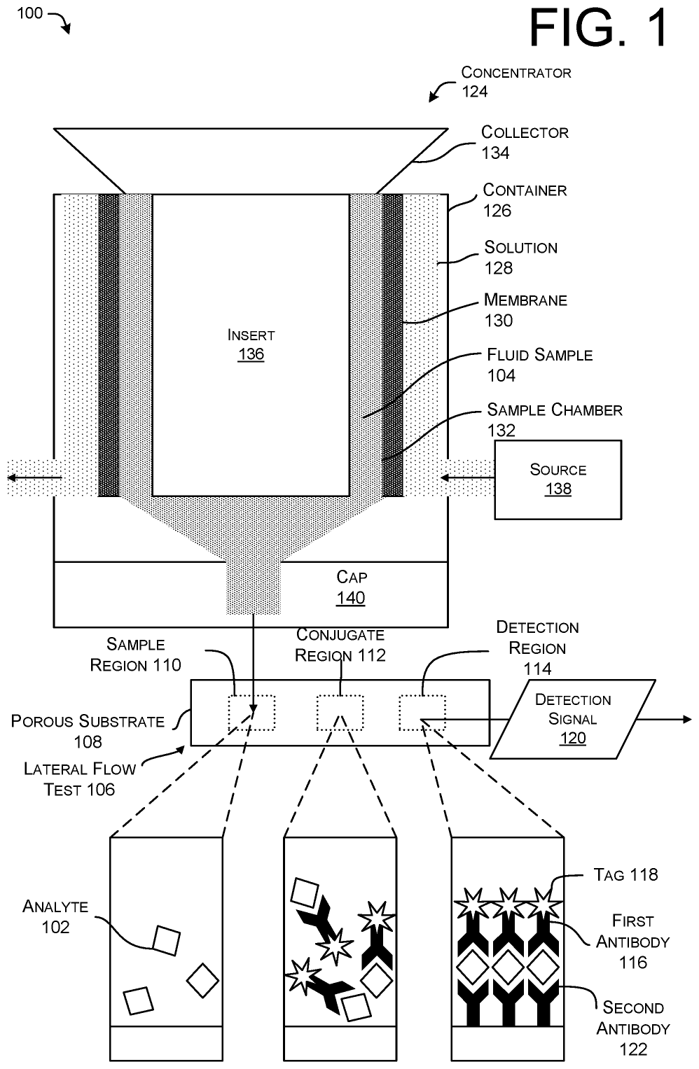

FIG. 1 illustrates an example system 100 for detecting the presence of an analyte 102 in a fluid sample 104. In various implementations, the fluid sample 104 is a biological sample. For instance, the fluid sample 104 includes at least one of urine, blood, saliva, serum, semen, mucus, or any other type of bodily fluid. According to some cases, the fluid sample 104 includes a solvent and a mucosal sample of a subject obtained by rubbing a nasopharyngeal swab inside of at least one nostril of the subject.

In various implementations, the analyte 102 includes a structure that is relevant to the health of the subject from which the fluid sample 104 is obtained. The subject, for instance, may be a human being or another type of animal. According to some implementations, the analyte 102 includes a structure that causes an infectious disease. As used herein, the term “infectious disease,” and its equivalents, can refer to a disorder caused by an infectious agent (e.g., bacteria, viruses, fungi, parasites, or a combination thereof). Examples of the infectious disease include coronavirus-based infections, such as middle east respiratory syndrome (MERS), severe acute respiratory syndrome (SARS), and coronavirus disease 19 (COVID-19); Corynebacterium-based infections, such as diphtheria; ebolavirus-based infections, such as ebola; orthomyxoviridae virus-based infections, such as influenza A, B, or C; hepatovirus A, B, C, D, or E-based infections, such as hepatitis; Haemophilus-based infections, such as hib disease; human immunodeficiency virus (HIV)-based infections, such as acquired immunodeficiency syndrome (AIDS); human papillomavirus (HPV)-based infections; Morbillivirus-based infections, such as measles; Mycobacterium-based infections, such as tuberculosis; Neisseria-based infections, such as meningitis; Orthorubulavirus-based infections, such as mumps; norovirus-based infections; Streptococcus-based infections; enterovirus-based infections, such as polio; Orthopneumovirus-based infections; rotavirus-based infections; Rubivirus-based infections, such as rubella; herpesvirus-based infections, such as chickenpox or shingles; Clostridium-based infections, such as tetanus or botulism; Bordatella-based infections, such as pertussis; Flavivirus-based infections, such as Zika; and so on. For instance, the analyte 102 may include at least one of a virus, a bacterium, or another type of cell of an infectious agent. Thus, the presence of the analyte 102 may be indicative of a disease experienced by the subject from which the fluid sample 104 is obtained.

In some implementations, the analyte 102 is one or more biomolecules, such as an amino acid, a protein, at least one nucleic acid (e.g., RNA and/or DNA), or a carbohydrate. In particular examples, the target is a biomolecule associated with an infectious agent, such as protein expressed on an outer surface of an infectious agent.

In some implementations, the analyte 102 is one or more nucleic acids sequence specific to the infectious agent. For example, the target is a viral RNA sequence of SARS-CoV-2, such as one or more sequences described in Sah et al., DOI: 10.1128/MRA.00169-20, Kames et al., SCIENTIFIC REPORTS 10 (Sep. 24, 2020), and Sah et al., MICROBIOLOGY RESOURCE ANNOUNCEMENTS 9(11) (2020).

In some cases, the analyte 102 includes at least a portion of a gene. As used herein, the term “gene” refers to a nucleic acid sequence that encodes a targeting agent. This definition includes various sequence polymorphisms, mutations, and/or sequence variants wherein such alterations do not affect the function of the encoded targeting agent. The term “gene” may include not only coding sequences but also regulatory regions such as promoters, enhancers, and termination regions. The term further can include all introns and other DNA sequences spliced from an mRNA transcript, along with variants resulting from alternative splice sites. Nucleic acid sequences encoding the targeting agent can be DNA or RNA that directs the expression of the targeting agent. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein.

As used herein, the term “encoding” refers to a property of sequences of nucleotides in a polynucleotide, such as a plasmid, a gene, cDNA, mRNA, to serve as templates for synthesis of targeting agents. A polynucleotide can, e.g., encode a protein if transcription and translation of mRNA produced by that gene produces the protein in a cell or other biological system. Unless otherwise specified, polynucleotides having a sequence encoding a targeting agent include all nucleotide sequences that are degenerate versions of each other and that encode the same amino acid sequence. The polynucleotides that encode proteins and RNA can also include introns. In some embodiments, the analyte 102 includes a plasmid, a cDNA, or an mRNA that can include, e.g., a sequence (e.g., a gene). According to some cases, the analyte 102 includes at least one biomarker for cancer.

In various cases, a lateral flow test (LFT) 106 is configured to detect the presence of the analyte 102 in the fluid sample 104. As used herein, the terms “lateral flow test,” “LFT,” “lateral flow assay test,” and “LFA” can be used interchangeably. The LFT 106 includes a porous substrate 108 that is configured to move the fluid sample 104 from a sample region 110, to a conjugate region 112, and into a detection region 114. Examples of a material in the porous substrate 108 include a cellulose and/or a cellulose-based material (e.g., cellulose acetate, cellulose triacetate, cellulose propionate, cellulose acetate propionate, cellulose acetate butyrate, cellulose sulfate, nitrocellulose, methylcellulose, ethylcellulose, ethyl methyl cellulose, hydroxyethyl cellulose, hydroxyethyl methyl cellulose, ethyl hydroxyethyl cellulose, carboxymethyl cellulose, etc.), cotton, nylon, a fluorinated polymer (e.g., polytetrafluoroethylene), polypropylene, fiberglass, metal, or any combination thereof. The porous substrate 108 may include a woven material, a non-woven material, a molded structure, a fabric, a foam a fibrous web, a paper, or any combination thereof.

In various implementations, the porous substrate 108 includes pores. For instance, the pores may have diameters in a range between 0.05 to 12 μm. In some cases, when a fluid is disposed on a first portion of the porous substrate 108, the fluid spontaneously moves into a second portion of the porous substrate 108. The fluid moves spontaneously due to capillary action via pores and/or capillaries in the porous substrate 108, in various implementations. In other words, the fluid can move laterally along the porous substrate 108 via wicking.

The LFT 106 includes a first antibody 116 that is disposed on the porous substrate 108 at the conjugate region 112. The first antibody 116, in some cases, is chemically and/or electrostatically bound to the porous substrate 108. For example, the first antibody 116 is printed and/or electroblotted onto the porous substrate 108. In some cases, the first antibody 116 is printed (e.g., ink-jet printed) onto the porous substrate 108. In various cases, the first antibody 116 is attached to a tag 118 that is configured to output a detection signal 120. When the fluid sample 104 moves into the conjugate region 112, the first antibody 116 specifically binds to the analyte 102.

The LFT 106 further includes a second antibody 122 that is disposed on the porous substrate 108 at the detection region 114. The second antibody 122 may be chemically and/or electrostatically bound to the porous substrate 108. When the fluid sample 104 moves into the detection region 114, carrying the analyte 102 bound to the first antibody 116, the second antibody 122 specifically binds to the analyte 102. In various implementations, the tag 118 outputs the detection signal 120 at the detection region 114. For instance, the tag 118 may include a fluorescent tag (e.g., a fluorescent dye, a fluorescent nanoparticle, a quantum dot, etc.), colloidal gold, a carbon label, a carbon nanotube, or any combination thereof.

The first antibody 116 and the second antibody 122 may specifically bind to the analyte 102. For instance, the first antibody 116 and the second antibody 122 may include one or more binding domains, such as cell marker ligands, receptor ligands, antibodies, peptides, peptide aptamers, nucleic acids, nucleic acid aptamers, Spiegelmers® L-nucleotid nucleic acids, or combinations thereof.

“Antibodies,” as used herein, are one example of binding domains and include whole antibodies or binding fragments of an antibody, e.g., Fv, Fab, Fab′, F(ab′)2, Fc, and single chain Fv fragments (scFvs) or any biologically effective fragments of an immunoglobulin that bind specifically to a motif expressed by a lymphocyte. In various implementations, any antibodies described herein can be replaced with other types of structures with binding domains, unless otherwise specified. Antibodies or antigen-binding fragments include all or a portion of polyclonal antibodies, monoclonal antibodies, human antibodies, humanized antibodies, synthetic antibodies, chimeric antibodies, bispecific antibodies, mini bodies, and linear antibodies.

Antibodies that specifically bind a motif expressed by a lymphocyte can be prepared using methods of obtaining monoclonal antibodies, methods of phage display, methods to generate human or humanized antibodies, or methods using a transgenic animal or plant engineered to produce antibodies as is known to those of ordinary skill in the art. Phage display libraries of partially or fully synthetic antibodies are available and can be screened for an antibody or fragment thereof that can bind to a lymphocyte motif. For example, binding domains may be identified by screening a Fab phage library for Fab fragments that specifically bind to a target of interest (see Hoet et al., NAT. BIOTECHNOL. 23:344, 2005). Phage display libraries of human antibodies are also available. Additionally, traditional strategies for hybridoma development using a target of interest as an immunogen in convenient systems (e.g., mice, HuMAb Mouse®, llamas, chicken, rats, hamsters, rabbits, etc.) can be used to develop binding domains. In particular embodiments, antibodies specifically bind to motifs expressed by a selected lymphocyte and do not cross react with nonspecific components or unrelated targets. Once identified, the amino acid sequence or polynucleotide sequence coding for the antibody can be isolated and/or determined.

Peptide aptamers include a peptide loop (which is specific for a target protein) attached at both ends to a protein scaffold. This double structural constraint greatly increases the binding affinity of the peptide aptamer to levels comparable to an antibody. The variable loop length is typically 8 to 20 amino acids (e.g., 8 to 12 amino acids), and the scaffold may be any protein which is stable, soluble, and small (e.g., thioredoxin-A, stefin A triple mutant, green fluorescent protein, eglin C, and cellular transcription factor Spl). Peptide aptamer selection can be made using different systems, such as the yeast two-hybrid system (e.g., Gal4 yeast-two-hybrid system) or the LexA interaction trap system.

Nucleic acid aptamers are single-stranded nucleic acid (DNA or RNA) ligands that function by folding into a specific globular structure that dictates binding to target proteins or other molecules with high affinity and specificity, as described by Osborne et al., CURR. OPIN. CHEM. BIOL. 1:5-9, 1997; and Cerchia et al., FEBS Letters 528:12-16, 2002. In particular embodiments, aptamers are small (˜15 KD; or between 15-80 nucleotides or between 20-50 nucleotides). Aptamers are generally isolated from libraries consisting of 1014-1015 random oligonucleotide sequences by a procedure termed SELEX (systematic evolution of ligands by exponential enrichment; see, for example, Tuerk et al., Science, 249:505-510, 1990; Green et al., METHODS ENZYMOLOGY. 75-86, 1991; and Gold et al., ANNU. REV. BIOCHEM., 64: 763-797, 1995). Spiegelmers® are similar to nucleic acid aptamers except that at least one β-ribose unit is replaced by β-D-deoxyribose or a modified sugar unit selected from, for example, β-D-ribose, α-Dribose, β-L-ribose.

Thus, the LFT 106 is configured to detect the presence of the analyte 102. However, in various cases, the fluid sample 104, as collected from the subject, includes the analyte 102 at a relatively low concentration. The low concentration may be below the limit of detection (LOD) of the LFT 106. Thus, if the as-collected fluid sample 104 is placed on the sample region 110 of the LFT 106, the detection signal 120 may be too weak to be detected. For example, if the detection signal 120 is a color-change of the detection region 114, the color change resulting from the presence of the analyte 102 in the as-collected fluid sample 104 may not be discernible to the naked eye. In other words, the concentration of the analyte 102 in the as-collected fluid sample 104 is below the LOD of the lateral flow test 106.

Although not specifically illustrated in FIG. 1, another type diagnostic assay can be substituted for the LFT 106. For example, an immunoassay, a mass spectrometer, or a polymerase chain reaction (PCR) system configured to detect the analyte 102 may be substituted for the LFT 106. However, the LOD of the other type of diagnostic assay may nevertheless exceed the concentration of the analyte 102 in the as-collected fluid sample 104.

In various implementations, a concentrator 124 is configured to increase the concentration of the analyte 102 in the fluid sample 104 before the fluid sample 104 is detected using the LFT 106 (or other diagnostic assay). In various cases, the concentrator 124 increases the concentration of the analyte 102 in the fluid sample 104 to a level that is greater than the LOD of the LFT 106. In addition, the concentrator 124 may passively concentrate the analyte 102 in the fluid sample 104. That is, the concentrator 124 can increase the concentration of the analyte 102 in the fluid sample 104 without requiring the use of a battery or other power source. Thus, in some examples, the concentrator 124 is suitable for particular low-resource settings.

The concentrator 124 may include a container 126 that is configured to at least partially enclose a solution 128 and a membrane 130. The solution 128 includes at least one first solute. The first solute(s) include, for instance, at least one of polyethylene glycol (PEG), polystyrene sulfonate (PSS), polyacrylic acid (PAA), polyethyleneimine (PEI), pectin, one or more detergents (e.g., sodium dodecyl sulfate (SDS), a polymer surfactant, an amphiphilic polymer, Pluronic, etc.), or any combination thereof. In various implementations, the first solute(s) include at least one polymer (also referred to as a “polymeric substance”). In some implementations, a molecular weight of the solute(s) in the solution 128 are greater than or equal to a threshold, such as 500 Daltons (Da), 1,000 Da, 1,500 Da, 5,000 Da, 10,000 Da, 100,000 Da, or 1,000,000 Da. For instance, the solute(s) in the solution 128 may have a molecular weight in a range of 1,000 to 1,000,000 Da. According to some implementations, the first solute(s) include micelles or other self-assembling structures. For instance, the solution 128 includes a polar solvent (e.g., water), a detergent (e.g., SDS), and a hydrophobic material (e.g., oil), wherein the detergent self-assembles into micelles into the solution 128 around the hydrophobic material.

In some examples, the concentration of the first solute(s) in the solution 128 are greater than or equal to 10 milligrams per milliliter (mg/mL), 50 mg/mL, 100 mg/mL, 500 mg/mL, 1 gram (g)/mL, 2 g/mL, or 5 g/mL. For instance, the concentration of the first solute(s) is in a range of 20 mg/mL to 3 g/mL. In various implementations, the concentration of the first solute(s) in the solution 128 is greater than the concentration of one or more second solutes (which may, in some cases, include the analyte 102) in the as-collected fluid sample 104. In some cases, the concentration of the first solute(s) in the solution 128 is greater than the LOD of the LFT 106. For example, the concentration of TB LAM in a urine sample may be 15 pg/mL, whereas an LFT 106 configured to detect TB LAM may have an LOD in a range of 500-1,000 pg/mL. As applied to the example of FIG. 1, the concentration of the first solute(s) in the solution 128 may be greater than the LOD of 1,000 pg/mL, such that the solution 128 may be configured to concentrate the fluid sample 104 to a level that is greater than the LOD of the LFT 106.

The membrane 130 may be semi-permeable. In various cases, pores extending through the membrane 130 may be large enough such that a fluid in the fluid sample 104 (e.g., water) can pass through the pores, but may be small enough such that the analyte 102 and the solute(s) in the solution 128 cannot pass through the pores. For example, the pores may have a diameter in a range of 2 to 100 nanometers (nm). In various implementations, the membrane 130 includes cellulose. For instance, the membrane 130 includes a dialysis membrane. In some examples, the membrane 130 is flexible (e.g., elastic and/or viscoelastic). In various implementations, the membrane 130 separates the solution 128 from a sample chamber 132 in the container 126. The solution 128 may be disposed between the membrane 130 and an inner sidewall of the container 126.

Although not specifically illustrated in FIG. 1, in some cases, a membrane holder is configured to hold the membrane 130 in a predetermined three-dimensional (3D) shape. According to various implementations, the membrane holder includes a web, lattice, or cage that includes a relatively inflexible material, such as polystyrene, stainless steel, polyethylene terephthalate, nylon, ABS, polypropylene, polyurethane, polyetherimide, polyphenol sulfide, or another material that is impervious and impermeable to the fluid sample 104 and the solution 128. At least a portion of the membrane holder may be disposed against the membrane 130. Thus, in cases wherein the membrane 130 is flexible, the membrane holder may cause the membrane 130 to maintain the predetermined shape.

According to various examples, the as-collected fluid sample 104 is deposited in the sample chamber 132. In some examples, the concentrator 124 includes a collector 134 that directs the fluid sample 104 into the sample chamber 132. For instance, the collector 134 includes a funnel. In various implementations, the concentrator 124 further includes a filter, which may include a porous substrate that is configured to capture precipitate in the fluid sample 104 before the fluid sample 104 enters the sample chamber 132.

In the example illustrated in FIG. 1, when the fluid sample 104 is disposed inside of the sample chamber 132, the membrane 130 is disposed between the fluid sample 104 and the solution 128. However, in some configurations, the membrane 130 may enclose a space in which the solution 128 is disposed, and the fluid sample 104 may be disposed between the membrane 130 and the inner sidewall of the container 126. In some cases, multiple instances of the membrane 130 and volumes of the solution 128 are disposed in the collector 132.

At least one solvent is at least partially removed from the fluid sample 104 by the solution 128. For example, a discrepancy between the concentration of second solute(s) in the fluid sample 104 and the concentration of the first solute(s) in the solution 128 induces an osmotic pressure between the solution 128 than the fluid sample 104. Due to the osmotic pressure, the solvent(s) in the fluid sample 104 spontaneously flows out of the fluid sample 104 and into the solution 128 through the pores in the membrane 130.

In some examples, an insert 136 is disposed inside of the sample chamber 132. In various implementations, the insert 136 includes a solid material that is impermeable to the fluid sample 104. For instance, the insert 136 may include a 3D shape that includes polystyrene, stainless steel, or some other material that is impervious to the fluid sample. In various cases, the insert 136 may be removeable from and/or insertable into the sample chamber 132. The insert 136, in some cases, increases the hydrostatic pressure of the fluid sample 104, which can further increase the flow rate of the solvent(s) from the fluid sample 104 to the solution 128. In some cases, the insert 136 increases the area at which the fluid sample 104 contacts the membrane 130, which further increases the flow rate of the solvent(s) from the fluid sample 104 to the solution 128. In various implementations, with or without the insert, the ratio of the area of the membrane in contact with the fluid sample 104 to the volume of the fluid sample 104 itself may be in a range of 0.1 to 100 centimeters squared per milliliter (cm²/mL), such as in a range of 1 to 10 cm²/mL. In some implementations, the insert 136 includes a membrane (similar to the membrane 130) that separates the sample chamber 132 from an additional volume of the solution 128, such that the insert 136 also pulls the solvent(s) from the fluid sample 104 into the additional volume of the solution 128.

In some cases, the flow rate of the solvent(s) from the fluid sample 104 to the solution 128 can be further increased by a source 138 configured to indue a flow of the solution 128 into and out of the container 126. The source 138 may transform the removal of the solvent(s) from the fluid sample 104 from a batch process to a continuous process. As the solvent(s) move from the fluid sample 104 into the solution 128, the solute(s) in the solution 128 may become diluted, which can decrease the osmotic pressure over time. To maintain the osmotic pressure, the diluted solution 128 can be replaced with undiluted solution 128 by the source 138. In some implementations, the source 138 cycles the solution 128 through the sample chamber 132. In some cases, the source 138 includes a reservoir from which the source 138 injects unused solution 128 into the container 126. In various implementations, the source 138 includes a pump, such as a peristaltic pump, that generates the flow of the solution 128 into and/or out of the container 126.

According to various implementations, the container 126 is connected to a cap 140. The cap 140 may define a space that is fluidly coupled to the sample chamber 132. In some cases, the space defined by the cap 140 is isolated from the solution 128. The space in the cap 140, for example, has a defined volume (e.g., 100 μL). In some examples, the space in the cap 140 can be isolated from the sample chamber 132, such as by removing the cap 140 from the container 126.

Although not specifically illustrated in FIG. 1, in some implementations, the cap 140 is integrated with the collector 134. For example, after loading the fluid sample 104 into the sample chamber 132, the cap 140 may be physically coupled to the collector 134. Optionally, the concentrator 124 is flipped over after the fluid sample 104 is loaded, such that the fluid sample 104 is drawn into the cap 140 by gravity.

In various implementations, the removal of the solvent(s) from the fluid sample 104 decreases a pressure (e.g., air pressure) within the sample chamber 132 if the sample chamber 132 is fluidically sealed. This low pressure can prevent further solvent(s) from leaving the sample chamber 132 and entering the solution 128. To prevent the low pressure, in some cases, the sample chamber 132 is vented to an external space (e.g., the atmosphere). For instance, at least one channel (also referred to as a “vent”) may extend between the sample chamber 132 and an exterior space outside of the concentrator 124 (e.g., atmosphere) through the container 126. In some implementations, a material is disposed in the channel(s) that seals the fluid sample 104 in the sample chamber 132 while enabling an exchange of gasses between the sample chamber 132 and the external atmosphere. The material, in various cases, is permeable to air, but is impermeable to the fluid sample 104. For instance, a polytetrafluoroethylene (PTFE) membrane, or other hydrophobic membrane, is disposed in the channel(s). Thus, the material may prevent a decrease in pressure in the sample chamber 132 while also preventing the fluid sample 104 from leaking from the concentrator 124.

In some cases, the solution 128 is disposed in a solution chamber within the concentrator 124. In various implementations, the removal of the solvent(s) from the fluid sample 104 increases a pressure (e.g., air pressure) within solution chamber if the solution chamber is fluidically sealed. This high pressure can prevent further solvent(s) from leaving the sample chamber 132 and entering the solution 128. To prevent the high pressure, in some cases, the solution chamber is vented to an external space (e.g., the atmosphere). For instance, at least one channel may extend between the solution chamber and an exterior space outside of the concentrator 124 (e.g., atmosphere) through the container 126. In some implementations, a material is disposed in the channel(s) that seals the solution 128 in the container 126 while enabling an exchange of gasses between the solution chamber and the external atmosphere.

Once the analyte 102 in the fluid sample 104 has been sufficiently concentrated such that the concentration of the analyte 102 exceeds the LOD of the LFT 106, the volume of the fluid sample 104 in the space of the cap 140 can be deposited on the LFT 106. In some implementations, the volume is manually transferred from the cap 140 to the LFT 106, such as via a dropper or pipet. In some cases, the sample region 110 of the LFT 106 is in contact with the space itself, or a channel that connects the space of the cap 140 to the sample region 110. Due to the heightened concentration of the analyte 102 in the fluid sample 104, the detection signal 120 (e.g., a color change of a portion of the LFT 106) output by the tag 118 in the LFT 106 is more easily discerned.

Particular implementations of the present disclosure enable the LFT 106 to the analyte 102, even when the LFT 106 is not sensitive enough to enable detection of the analyte 102 in the as-collected fluid sample 104. In various cases, the concentrator 124 sufficiently concentrates the fluid sample 104 to enable detection of the analyte 102 using the LFT 106 without requiring the use of significant energy into the system 100. For example, the removal of the solvent(s) from the fluid sample 104 can be performed passively, such as without the use of a battery, pump, or other type of active element.

In various cases, the concentrator 124 concentrates the analyte 102 in the fluid sample 104 to a level that is above the LOD of the LFT 106 within a relatively short amount of time. For example, the concentrator 124 may sufficiently concentrate the fluid sample 104 in a time period that is within a range of 1 to 60 minutes, a range of 10 to 30 minutes, or the like. For instance, the concentrator 124 may sufficiently concentrate the fluid sample 104 in 15 minutes after the fluid sample 104 is deposited in the concentrator 124.

Although the sufficiency of the concentration of the analyte 102 in the fluid sample 104 may vary depending on the LOD of the LFT 106 (or alternate diagnostic assay), in various cases, the concentrator 124 may concentrate the analyte 102 in the fluid sample 104 by 5, 10, 50, 100, 200, or 500 times. For example, the concentration of the analyte 102 in the concentrated fluid sample 104 may be 100 times the concentration of the analyte 102 in the fluid sample 104 as-obtained from the subject.

A particular example will now be described with reference to FIG. 1. A nasopharyngeal swab is used to obtain a mucus sample from the interior of a subject's nose. The swab is disposed in a vessel containing a test solvent. The fluid sample 104 is generated when the mucus sample is mixed with the test solvent. The LFT 106 may be an antigen point-of-care test (AgPOCT) configured to detect the presence of SARS-CoV-2 nucleocapsid (N) protein. However, the subject may have a relatively small viral load, such that the presence of the N protein in the fluid sample 104 cannot be reasonably detected by the LFT 106. In other words, the concentration of the N protein in the fluid sample is below the LOD of the LFT 106.

The concentrator 124 receives the fluid sample 104. For instance, the fluid sample 104 is poured into the sample chamber 132 via the collector 134. After the fluid sample 104 is poured into the sample chamber 132, the insert 136 is placed in the sample chamber 134 in order to increase the interior surface area of the membrane 130 that is in contact with the fluid sample 104.

The solution 128 includes at least one first solute, and the fluid sample 104 includes at least one second solute. The first solute(s), for example, include PEG with a molecular weight of 1,500 Da. The concentration of the first solute(s) in the solution 128 is significantly higher than the concentration of the second solute(s) in the fluid sample 104. Accordingly, water (and optionally, other hydrophilic solvents in the fluid sample 104) spontaneously moves from the fluid sample 104, through the membrane 130, and into the solution 128. Because the pores in the membrane 130 are smaller than the N protein, the movement of the solvent(s) out of the fluid sample 104 concentrates the N protein in the fluid sample 104.

After a predetermined time period (e.g., 15 minutes), the fluid sample 104 is sufficiently concentrated. That is, the concentration of the N protein in the fluid sample 104 is now higher than the LOD of the LFT 106. After concentration is performed, a predetermined volume (e.g., 100 μL) of the fluid sample 104 is disposed in the space defined by the cap 140. In various cases, the volume is transferred to the sample region 110 of the LFT 106. As capillary action moves the fluid sample 104 along a length of the porous substrate 108, the N protein is bound to the first antibody 116 in the conjugate region 112, and the second antibody 122 in the detection region 114. Due to the high concentration of the N protein in the concentrated fluid sample 104, a visual signal (e.g., a color change of a strip shape in the detection region 114) is output as the detection signal 120. In some cases, the detection signal 120 is visible to the user. In various examples, the detection signal 120 is detected by a sensor of a device (e.g., a camera of a mobile phone), and the device outputs an indication to the user that the N protein is present in the fluid sample 104. Accordingly, even though the viral load of the subject is below the LOD of the LFT 106, the concentrator 124 can be used to accurately determine that the subject has a SARS-CoV-2 infection.

Although FIG. 1 has been described with reference to concentrating a biological specimen, implementations are not so limited. Implementations of the present disclosure can be utilized to concentrate various types of targets-of-interest in a fluid sample. For instance, an industrial process may generate a valuable biologic (e.g., an antibody therapy) in a fluid sample 104. To obtain the valuable biologic in a concentrated format, the solvent from the fluid sample 104 may be extracted using the concentrator 124. In this example, the concentrated fluid sample 104 can be utilized for therapeutic effect, rather than for diagnostic purposes.

In addition, although FIG. 1 illustrates that the solution 128 is disposed around the fluid sample 104, implementations are not so limited. In some examples, the fluid sample 104 may be disposed between the membrane 130 and a sidewall of the container 126, and the membrane 130 may at least partially enclose the solution 128. Other physical orientations of these constituent elements are also possible.

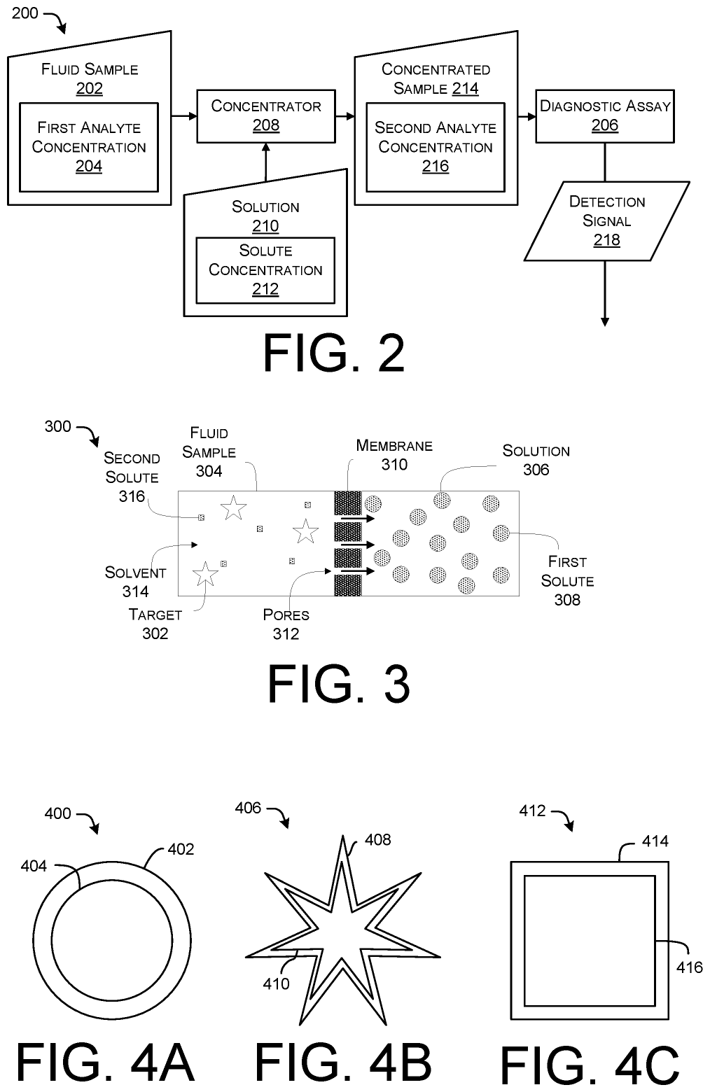

FIG. 2 is a diagram 200 illustrating an example flow of materials and components for concentrating a fluid sample 202. In some cases, the fluid sample 202 includes blood, urine, mucus, semen, saliva, or some other type of biological specimen obtained from a subject. In some examples, the fluid sample 202 includes an industrial batch of a biologic or other therapeutically significant material. The fluid sample 202 includes a first analyte concentration 204. The analyte, for instance, may include at least one of tuberculosis antigen LAM, HIV, hGH, cell-free DNA, hCG, SARS-CoV-2 nucleocapsid (N) protein, an exosome, RNA, a polysaccharide, a bacterium, or a virus. A diagnostic assay 206 may be configured to detect the analyte. However, in various cases, the first analyte concentration 204 may be smaller than a LOD of the diagnostic assay 206. The diagnostic assay 206, for instance, may be an LFT.

The fluid sample 202 may be concentrated using a concentrator 208. In various cases, the concentrator 208 may include, or otherwise utilize, a solution 210 that includes a solute concentration 212. In various cases, the concentrator 208 includes a porous membrane that is disposed between the fluid sample 202 and the solution 210. The solution 210 includes one or more solutes, such as polymers, detergents, or a combination thereof. The solute concentration 212 is sufficiently high to spontaneously induce movement of a solute (e.g., water) from the fluid sample 202 into the solution 210 through the porous membrane. That is, the solute concentration 212 is greater than a concentration of at least one solute (which may include the analyte) in the fluid sample 202.

In various implementations, the concentrator 208 outputs a concentrated sample 214. The concentrated sample 214 may be the fluid sample 202 with at least a portion of the solute removed. The portion of the solute may remain in the solution 210, for instance. In various cases, the concentrated sample 214 has a second analyte concentration 216. The second analyte concentration 216 is greater than the first analyte concentration 204. In various implementations, the second analyte concentration 216 exceeds the LOD of the diagnostic assay 206.

In various implementations, the diagnostic assay 206 is used to detect the analyte in the concentration sample 214. In various implementations, the diagnostic assay 206 outputs a detection signal 218 that is indicative of the presence of the analyte in the fluid sample 202 and the concentrated sample 214. For example, the detection signal 218 may be a visual signal that indicates the presence of the analyte in the fluid sample 202.

FIG. 3 illustrates an example environment 300 for concentrating a target 302 in a fluid sample 304. In some cases, the target 302 is an analyte, such as the analyte 102 described above with reference to FIG. 1. In some cases, the target 302 includes a biological molecule or structure that is concentrated for a non-diagnostic reason. For instance, the target 302 may include at least one of an antibody, an antigen, a protein, RNA, DNA, a carbohydrate, a micelle, a deactivated virus, a biopharmaceutical, or some other biological structure with therapeutic effect. In some cases, the target 302 is concentrated as part of a manufacturing process. In some implementations, the target 302 is not a biological molecule or structure. For example, the target 302 may include a nanoparticle (e.g., a magnetic nanoparticle, quantum dot, or the like). In various cases, the fluid sample 304 includes the fluid sample 104 described above with reference to FIG. 1.

The concentration of the target 302 in the fluid sample 304 may be increased using a solution 306. The solution 306 may include a first solute 308. The first solute 308 is present in the solution 306 at a first concentration. In some cases, the first solute 308 includes a polymer, but implementations are not so limited. In some cases, the first solute 308 includes micelles that are spontaneously generated using a detergent or other amphipathic molecule.

As illustrated in FIG. 3, a membrane 310 is disposed between the fluid sample 304 and the solution 306. In some cases, the membrane 310 includes the membrane 130 described above with reference to FIG. 1. Various pores 312 extend through the membrane 310. In various cases, the pores 312 fluidly connect the fluid sample 304 to the solution 306. In some cases, the membrane 310 includes cellulose.

According to various implementations, the target 302 is concentrated in the fluid sample 304 by removing a solvent 314 from the fluid sample 304. In various cases, the solvent 314 includes water and/or a hydrophilic solvent (e.g., an alcohol). According to various implementations, the fluid sample 304 includes a second solute 316 that is dissolved in the solvent 314. In some cases, the target 302 is also dissolved in the solvent 314, but implementations are not so limited. According to some cases, the target 302 is the second solute 316. The second solute 316 is present in the fluid sample 304 at a second concentration.

In various examples, the solvent 314 spontaneously flows from the fluid sample 304 into the solution 306 through the pores 312 due to osmotic pressure. For instance, the first concentration of the first solute 308 in the solution 306 is higher than the second concentration of the second solute 316 in the fluid sample 304. As a result of the mismatch in the first and second concentrations, the solvent 314 preferentially flows into the solution 306. Due to the movement of the solvent 314 out of the fluid sample 304, the concentration of the target 302 (and the second solute 316) in the fluid sample 304 increases. Various implementations can be therefore utilized to increase the concentration of the target 302 in the fluid sample 304.

FIGS. 4A to 4C illustrate cross-sections of various concentrators in accordance with implementations of the present disclosure. FIG. 4A illustrates a cross-section of a circular example concentrator 400 including a chamber wall 402 and a membrane 404. As shown, the cross-sections of the chamber wall 402 and the membrane 404 are circular. For instance, the chamber wall 402 and/or the membrane 404 may be cylindrical.

FIG. 4B illustrates a cross-section of an example concave concentrator 406 including a chamber wall 408 and a membrane 410. As shown, the cross-sections of the chamber wall 408 and the membrane 404 each have concave shapes. These concave shapes can produce a greater surface-to-volume ratio of the membrane and a fluid sample and/or solution used to concentrate the fluid sample than the circular shapes illustrated in FIG. 4A. In some implementations, the enhanced surface-to-volume ratio can be used to more rapidly concentrate an analyte in the fluid sample.

FIG. 4C illustrates a cross-section of an example convex concentrator 412 including a chamber wall 414 and a membrane 416. As shown, the cross-sections of the chamber wall 414 and the membrane 416 each have convex shapes. In some cases, these convex shapes have enhanced structural integrity and are easier to manufacture than more complex shapes.

In various cases, the concentrators 400, 406, and 412 are shaped like rectangular prisms, although implementations are not so limited. In various cases, a fluid sample and a highly concentrated solution are disposed inside of an example concentrator 400, 406, or 412. For instance, the fluid sample is disposed between the membrane 404, 410, or 416 and the chamber wall 402, 408, or 414, and the solution is disposed in an interior space of the membrane 404, 410, or 416. Alternatively, the solution is disposed between the membrane 404, 410, or 416 and the chamber wall 402, 408, or 414, and the fluid sample is disposed in an interior space of the membrane 404, 410, or 412. According to various implementations, the membrane 404, 410, or 416 is disposed between the fluid sample and the solution, such that a solute from the fluid sample is drawn through the membrane 404, 410, or 416 into the solution via osmotic pressure.

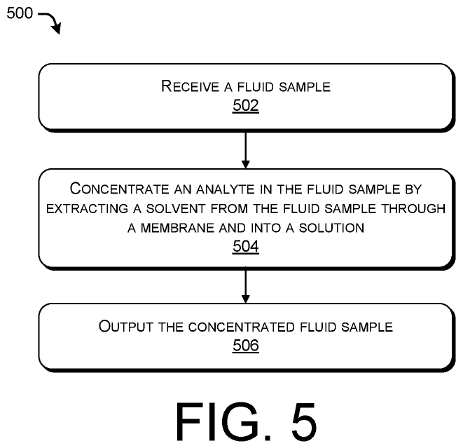

FIG. 5 illustrates an example process 500 for concentrating a fluid sample. The process 400 is performed by an entity, such as the concentrator 124, 208, 300, 306, 312, 400 described above with reference to FIGS. 1-4.

At 502, the entity receives a fluid sample. For example, the fluid sample may include at least one of urine, saliva, mucus, semen, blood, or some other biological specimen. In some cases, the fluid sample includes a mucosal sample obtained from a subject using a nasopharyngeal swab that has been swept in a nasal passage of the subject. The fluid sample may include an analyte. For instance, the fluid sample includes at least one of tuberculosis antigen LAM, HIV, hGH, cell-free DNA, hCG, SARS-CoV-2) protein, an exosome, RNA, a polysaccharide, a bacterium, or a virus. The concentration of the analyte in the fluid sample, as received by the entity, may be lower than a LOD of a diagnostic assay. In some implementations, the entity includes a funnel through which it receives the fluid sample

At 504, the entity concentrates an analyte in the fluid sample by extracting a solvent from the fluid sample through a membrane and into a solution. In various implementations, the membrane is disposed between the fluid sample and the solution. In some examples, the membrane includes cellulose. In various cases, the membrane is semi-permeable. For instance, water or other solvents can pass through pores in the membrane. In some cases, the diameters of the pores are greater than or equal to 2 nm and less than or equal to 100 nm.

The solution may include at least one first solute. Examples of the first solute(s) include at least one of PEG, PSS, PAA, PEI, pectin, or SDS. The first solute(s), for example, may include at least one of a polymer, a detergent, a surfactant, or a micelle. According to various examples, the concentration of the first solute(s) in the solution is relatively high. For instance, the concentration of the first solute(s) in the solution is greater than or equal to 0.2 g/mL and/or less than or equal to 3.0 g/mL. Further, in some cases, the first solute(s) have a relatively high molecular weight. For instance, the first solute(s) have a molecular weight that is greater than or equal to 500 Da and less than or equal to 1,000,000 Da. In various examples, the first solute(s) are smaller, in at least one dimension, than the diameters of the pores. The first solute(s) may be unable to pass through the pores in the membrane. In some cases, the solution may further include a solvent, such as water or some other hydrophilic solvent.

In some implementations, the membrane at least partially encloses an inner space, wherein the solution or the fluid sample is disposed in the inner space. In some implementations, the membrane is in the shape of a right prism, wherein a cross-section of the right prism is at least one of a circle, a concave polygon, or a convex polygon. To increase the surface-to-volume ratio of a surface area of the membrane with respect to the volume of the inner space, an insert may be further disposed in the inner space. The insert may be a physical object that pushes the fluid disposed in the inner space (i.e., the fluid sample or the solution) up the inner surface of the membrane, thereby increasing the amount of the surface area of the membrane that is in contact with the fluid in the inner space. The insert may be disposed at a distance of 0.5 mm to 20 mm from the membrane.

The entity may rapidly concentrate the analyte in the fluid sample. For example, the entity may concentrate the analyte in the fluid sample by 10 to 1,000 times in a particular time interval. In some cases, the particular time interval is 10 to 60 minutes. In various cases, the concentration of the analyte in the concentrated fluid sample may exceed the LOD of the diagnostic assay.

At 506, the entity outputs the concentrated fluid sample. In some examples, the concentrated fluid sample is output into a collection space defined by a collection cap. The collection space may have a predetermined volume. In some implementations, the entity outputs the concentrated fluid sample to the diagnostic assay, such that the diagnostic assay may be used to determine whether the analyte is present in the fluid sample.

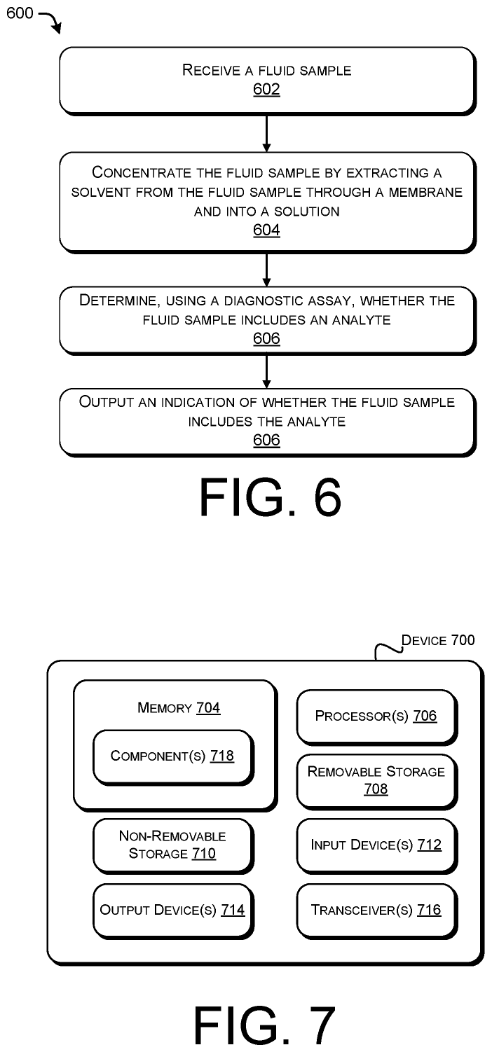

FIG. 6 illustrates an example process 600 for detecting an analyte in a fluid sample. The process 600 is performed by an entity, such as at least one of the LFT 106 or concentrator 124 described above with reference to FIG. 1 and/or at least one of a computing device or processor.

At 602, the entity receives a fluid sample. For example, the fluid sample may include at least one of urine, saliva, mucus, semen, blood, or some other biological specimen. In some cases, the fluid sample includes a mucosal sample obtained from a subject using a nasopharyngeal swab that has been swept in a nasal passage of the subject. The fluid sample may include an analyte. For instance, the fluid sample includes at least one of tuberculosis antigen LAM, HIV, hGH, cell-free DNA, hCG, SARS-CoV-2 N protein, an exosome, RNA, a polysaccharide, a bacterium, or a virus. The concentration of the analyte in the fluid sample, as received by the entity, may be lower than a LOD of a diagnostic assay. In some implementations, the entity includes a funnel through which it receives the fluid sample

At 604, the entity concentrates the fluid sample by extracting a solvent from the fluid sample through a membrane and into a solution. In various implementations, the membrane is disposed between the fluid sample and the solution. In some examples, the membrane includes cellulose. In various cases, the membrane is semi-permeable. For instance, water or other solvents can pass through pores in the membrane. In some cases, the diameters of the pores are greater than or equal to 2 nm and less than or equal to 100 nm.

The solution may include at least one first solute. Examples of the first solute(s) include at least one of PEG, PSS, PAA, PEI, pectin, or SDS. The first solute(s), for example, may include at least one of a polymer, a detergent, a surfactant, or a micelle. According to various examples, the concentration of the first solute(s) in the solution is relatively high. For instance, the concentration of the first solute(s) in the solution is greater than or equal to 0.2 g/mL and/or less than or equal to 3.0 g/mL. Further, in some cases, the first solute(s) have a relatively high molecular weight. For instance, the first solute(s) have a molecular weight that is greater than or equal to 500 Da and less than or equal to 1,000,000 Da. In various examples, the first solute(s) are smaller, in at least one dimension, than the diameters of the pores. The first solute(s) may be unable to pass through the pores in the membrane. In some cases, the solution may further include a solvent, such as water or some other hydrophilic solvent.

In some implementations, the membrane at least partially encloses an inner space, wherein the solution or the fluid sample is disposed in the inner space. In some implementations, the membrane is in the shape of a right prism, wherein a cross-section of the right prism is at least one of a circle, a concave polygon, or a convex polygon. To increase the surface-to-volume ratio of a surface area of the membrane with respect to the volume of the inner space, an insert may be further disposed in the inner space. The insert may be a physical object that pushes the fluid disposed in the inner space (i.e., the fluid sample or the solution) up the inner surface of the membrane, thereby increasing the amount of the surface area of the membrane that is in contact with the fluid in the inner space. The insert may be disposed at a distance of 0.5 mm to 20 mm from the membrane.

The entity may rapidly concentrate the analyte in the fluid sample. For example, the entity may concentrate the analyte in the fluid sample by 10 to 1,000 times in a particular time interval. In some cases, the particular time interval is 10 to 60 minutes. In various cases, the concentration of the analyte in the concentrated fluid sample may exceed the LOD of the diagnostic assay.

At 606, the entity determines, using the diagnostic assay, whether the fluid sample includes the analyte. The diagnostic assay, for example, may include a lateral flow assay, an immunoassay, a mass spectroscopy assay, a PCR assay, or a combination thereof. For instance, the concentrated fluid sample may be disposed on a porous substrate (e.g., paper) configured to move the concentrated fluid sample via capillary action. A first antibody and a second antibody, each of which specifically binds the analyte, may be disposed on the porous substrate. Due to the movement of the concentrated fluid sample on the porous substrate, the analyte may bind to both the first antibody and the second antibody. In some cases, the second antibody is bound to a tag (e.g., a dye) that emits a detection signal (e.g., a visual signal). According to various implementations, the detection signal is emitted by the diagnostic assay when the analyte is present in the concentrated fluid sample at a concentration that exceeds the LOD of the diagnostic assay.

At 608, the entity outputs an indication of whether the fluid sample includes the analyte. For instance, the detection signal itself may be the indication of whether the fluid sample includes the analyte. In some cases, the entity includes at least one sensor configured to detect the detection signal. In particular examples, the entity includes a camera that captures an image of the diagnostic assay (e.g., the porous substrate of the lateral flow assay), wherein the image visually captures the detection signal. The entity may further include a processor configured to determine, based on the detected detection signal, whether the analyte is present in the fluid sample. In various cases, the entity outputs an indication of the presence of the analyte to a user. For example, the entity may include a display, a speaker, or some other output device that outputs a signal indicating the presence of the analyte.

FIG. 7 illustrates an example of a device 700 for performing one or more functions described herein. The device 700 includes any of memory 704, processor(s) 706, removable storage 708, non-removable storage 710, input device(s) 712, output device(s) 714, and transceiver(s) 716. The device 700 may be configured to perform various methods and functions disclosed herein.

The memory 704 may include component(s) 718. The component(s) 718 may include at least one of instruction(s), program(s), database(s), software, operating system(s), etc. In some implementations, the component(s) 718 include instructions that are executed by processor(s) 706 and/or other components of the device 700.

In some embodiments, the processor(s) 706 include a central processing unit (CPU), a graphics processing unit (GPU), or both CPU and GPU, or other processing unit or component known in the art.

The device 700 may also include additional data storage devices (removable and/or non-removable) such as, for example, magnetic disks, optical disks, or tape. Such additional storage is illustrated in FIG. 7 by removable storage 708 and non-removable storage 710. Tangible computer-readable media can include volatile and non-volatile, removable and non-removable media implemented in any method or technology for storage of information, such as computer readable instructions, data structures, program modules, or other data. The memory 704, the removable storage 708, and the non-removable storage 710 are all examples of computer-readable storage media. Computer-readable storage media include, but are not limited to, Random Access Memory (RAM), Read-Only Memory (ROM), Electrically Erasable Programmable Read-Only Memory (EEPROM), flash memory, or other memory technology, Compact Disk Read-Only Memory (CD-ROM), Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device 700. Any such tangible computer-readable media can be part of the device 700.

The device 700 may be configured to communicate over a telecommunications network using any common wireless and/or wired network access technology. Moreover, the device 700 may be configured to run any compatible device Operating System (OS), including but not limited to, Microsoft Windows Mobile, Google Android, Apple iOS, Linux Mobile, as well as any other common mobile device OS.

The device 700 also can include input device(s) 712, such as a keypad, a cursor control, a touch-sensitive display, voice input device, etc. In some cases, the input device(s) 712 include a camera configured to capture an image of a diagnostic assay, such as an LFT, wherein the image is indicative of the presence of an analyte in a fluid sample. According to some implementations, the input device(s) 712 include at least one sensor configured to detect a detection signal (e.g., the detection signal 120 and/or 218) from a diagnostic assay, wherein the detection signal is indicative of the presence of the analyte in the fluid sample. In some cases, the processor(s) 706 analyze the image and/or detection signal in order to determine whether the analyte is present in the fluid sample. In various implementations, the output device(s) 714 include a display, speakers, printers, etc. For example, the output device(s) 714 may output an indication of whether the analyte is present in the fluid sample.

As illustrated in FIG. 7 the device 700 also includes one or more wired or wireless transceiver(s) 716. For example, the transceiver(s) 716 can include a network interface card (NIC), a network adapter, a Local Area Network (LAN) adapter, or a physical, virtual, or logical address to connect to various network components, for example. To increase throughput when exchanging wireless data, the transceiver(s) 716 can utilize multiple-input/multiple-output (MIMO) technology. The transceiver(s) 716 can comprise any sort of wireless transceivers capable of engaging in wireless, radio frequency (RF) communication. The transceiver(s) 716 can also include other wireless modems, such as a modem for engaging in Wi-Fi, WiMAX, Bluetooth, infrared communication, and the like. The transceiver(s) 716 may include transmitter(s), receiver(s), or both.

Various implementations will now be described with reference to the following examples. In various cases, features from one example can be applied to features from any other example, unless otherwise specified.

### First Example

Urine is an attractive biospecimen for in vitro diagnostics, and urine-based lateral flow assays are low-cost devices suitable for point-of-care testing, particularly in low-resource settings. However, some of the lateral flow assays exhibit limited diagnostic utility because the urinary biomarker concentration is significantly lower than the assay detection limit, which compromises the sensitivity. To address the challenge, an osmotic processor that statically and spontaneously concentrated biomarkers was developed. The specimen in the device interfaces with the aqueous polymer solution via a dialysis membrane. The polymer solution induces an osmotic pressure difference that extracts water from the specimen, while the membrane retains the biomarkers. The evaluation demonstrated that osmosis induced by various water-soluble polymers efficiently extracted water from the specimens, ca. 5-15 mL/hr. The osmotic processor concentrated the specimens to improve the lateral flow assays' detection limits for the model analytes-human chorionic gonadotropin and SARS-CoV-2 nucleocapsid protein. After the treatment via the osmotic processor, the lateral flow assays detected the corresponding biomarkers in the concentrated specimens. The test band intensities of the assays with the concentrated specimens were very similar to the reference assays with 100-fold concentrations. The mass spectrometry analysis estimated the SARS-CoV-2 nucleocapsid protein concentration increased ca. 200-fold after the osmosis. With its simplicity and flexibility, this device demonstrates a great potential to be utilized in conjunction with the existing lateral flow assays for enabling highly sensitive detection of dilute target analytes in urine.

### INTRODUCTION

Urine is one of the most used biospecimens next to blood that can be easily collected in large quantities with noninvasive procedures (HADLAND, S. E. et al., 2016. Child Adolesc. Psychiatr. Clin. N, Am., 25, 549-565). Urine is used at the point-of-care and in laboratory settings to detect pregnancies, diagnose diseases, and screen potential health problems (TUUMINEN, T. 2012. Front. Immunol., 3, 45). Molecules in urine originate from glomerular filtration of plasma, renal tubule excretion, and shedding of various cells, representing a biomarker repertoire that can be exploited for diagnosis and monitoring of renal as well as systemic diseases (HARPOLE, M., et al., 2016. Expert Rev, Proteomics, 13, 609-626). Urine is composed of mostly water and solutes like urea, small ions, creatinine, albumin, bilirubin, and low concentrations of other small proteins (SIMERVILLE, J. A., et al., 2005. Am. Fam. Physician, 71, 1153-1162; TAYLOR, E. N. et al., 2006. Am. J. Kidney Dis., 48, 905-915). Solute concentrations as well as the presence of other uncommon molecules are reflective of physiological conditions and can be utilized for disease diagnosis such as urinary tract infection (Simerville et al., 2005). However, urinary biomarkers can present in concentrations well below the limits of detection (LOD) of common diagnostic assays (NIMSE, S. B., et al., 2016. Analyst, 141, 740-755). For example, the concentration of human growth hormone (hGH), a urinary biomarker, is ca. 100-fold below the immunoassays' LOD (FREDOLINI, C., et al., 2008. Nano Res., 1, 502-518). Urinary cell-free DNA can be utilized as a biomarker for cancer and infectious disease diagnostics (e.g., tuberculosis), but the dilute concentration and fragmented nature of the analyte impair the efficiency of extraction methods and consequently lower the diagnostic sensitivity (ORESKOVIC, A., et al., 2019. J. Mol. Diagn., 21, 1067-1078). The presence of high salts and interfering molecules (e.g., biotin) in urine also hinders the development and clinical implementation of urine-based diagnostic tests (WONG, R. C. et al., 2009. Lateral flow immunoassay, New York, NY, Springer; BOWEN, R et al., 2019. Clin. Biochem., 74, 1-11).

Lateral flow assays (LFAs) are low-cost immunoassays for rapid biomarker detection, which have been widely used in medicine, environmental health, and quality control (KOCZULA, K. M. et al., 2016. Essays in biochemistry, 60, 111-120). However, the LFAs' detection limits are higher than the corresponding laboratory-based assays, so their sensitivities are significantly impacted by the low analyte concentrations and interferences (MOGHADAM, et al., 2015. Anal. Chem., 87, 1009-1017; ZHANG, Y. et al., 2020. Sci. Rep., 10, 9604). For example, the Alere Determine TBLAM Ag, a LFA for detecting urinary tuberculosis antigen lipoarabinomannan (LAM), has been proven to be highly specific and exhibits potential to be a high-impact point-of-care test (PETER, J., et al., 2015. BMC Infect. Dis., 15, 262). However, the estimated assay sensitivities are ˜18% for HIV-negative and ˜42% for HIV-positive individuals, caused by the low analyte concentration (BULTERYS, et al., 2019. J. Clin. Med. Res., 9). The LAM concentration in urine for TB-positive patients can be as low as 14 pg/mL, which is significantly lower than the assay LOD, 500-1000 pg/mL (Bulterys et al., 2019, GARCÍA, J. I., et al., 2019, Sci. Rep., 9, 18012). Thus, the test cannot be utilized for general TB screening (2020. Global tuberculosis report 2020. Geneva, Switzerland: World Health Organization).

Techniques have been developed to improve the sensitivities of LFAs, including kinetics and transport control (YANG, M. et al., 2013. Virol. J., 10, 125; RIVAS, L, et al., 2014. Lab Chip, 14, 4406-4414; TANG, R., et al., 2017. Sci. Rep., 7, 1360; ISHII, M., et al., 2018. Anal Sci, 34, 51-56), biochemical signal amplification (HU, J., et al., 2013. Lab Chip, 13, 4352-4357; PAROLO, C., et al., Biosens. Bioelectron., 40, 412-416; PANFEROV, V. G., et al., 2016. Talanta, 152, 521-30), improved labeling (CHOI, D. et al., 2010. Biosens Bioelectron, 25, 1999-2002), and sample enrichment. Sample enrichment techniques include centrifugal filtration (CORSTJENS, P. L, et al., 2015. Parasit. Vectors, 8, 241), immunomagnetic separation (BEN AISSA, A et al., 2021. Sensors (Basel), 21; PANFEROV, V. G., et al., 2017, Talanta, 164, 69-76), electrophoretic and phasic separation (WU, J. C. et al., 2014. Sensors, 14, 4399-4415; CHIU, R. Y., et al., 2015. PLoS One, 10, e0142654; KIM, C., et al., 2017. Lab Chip, 17, 2451-2458), isotachophoresis (Moghadam et al., 2015), dialysis (TANG, R., et al., 2016. Talanta, 152, 269-276), and test-zone pre-enrichment (Zhang et al., 2020). These systems are not suitable for low-resource, point-of-care settings as they require expensive reagents, equipment, and/or complex procedures. On the other hand, paper-based methods using dialysis or test-zone pre-enrichment lead to suboptimal enrichment and have limited processing capacity (Tang et al., 2016, Zhang et al., 2020). Therefore, there is a need for simple, versatile, and effective approaches that enrich analytes to improve the LFA sensitivity.

To enable sensitive and rapid urinary biomarker detection via LFAs, this experimental example includes results from testing an example concentrator, referred to as an osmotic processor-a device that concentrates analytes via osmosis. A process using the osmotic processor spontaneously removes water molecules from the urine specimen while retaining the target analyte. The device includes a urine specimen compartment and a polymer compartment, which are separated by a semipermeable membrane. The polymer solution induces a strong osmotic pressure difference across the semipermeable membrane, which drives water molecules in the urine specimen across the membrane into the polymer solution (NELSON, P. H. 2017. Eur. Biophys. J, 46, 59-64). Additionally, the membrane's molecular weight cutoff (MWCO) is significantly smaller than that of the target analyte, allowing small ions and solutes that may interfere with the assay to be removed. Concentrating urinary analytes via osmosis has been demonstrated by McFarlane, using cellulose acetate membrane and sucrose (polymer) to concentrate the analytes 5-fold for gel electrophoresis (MCFARLANE, H. 1964. Clin. Chin. Acta, 9, 376-380).

Compared to existing enrichment approaches, the osmotic processor demonstrates the potential to streamline its interface with existing LFAs, its simplicity with a spontaneous process, its flexibility to process a large specimen volume, and its capability to simultaneously recondition the concentrated specimen (remove inhibitory factors, which include molecules that can influence the assay results, thereby increasing the risk of producing a false positive or false negative result) for optimal assay performance. In this work the device was utilized to improve the detection limits of commercially available LFAs using human chorionic gonadotropin (hCG) and SARS-CoV-2 nucleocapsid (N) protein as model analytes. The osmotic processor has demonstrated ca. 100-fold concentration from a 10 mL sample for both analytes.

### Materials

Polyethylene glycol 1500/PEG 1500 (Sigma-Aldrich, St. Louis, MO, USA), Polyethylene glycol 4000/PEG 4000 (Sigma-Aldrich, St. Louis, MO, USA), Polyethylene glycol 35000/PEG 35000 (Sigma-Aldrich, St. Louis, MO, USA), Poly (sodium 4-styrenesulfonate)/PSS (Sigma-Aldrich, St. Louis, MO, USA), Pectin (Spectrum Chemical Mfg. Corp, Gardena, CA, USA), Poly(acrylic acid sodium salt)/PAA (Sigma-Aldrich, St. Louis, MO, USA), Polyethyleneimine/PEI (Sigma-Aldrich, St. Louis, MO, USA), Spectra/Por 1 Dry Standard Grade Regenerated Cellulose (RC) Dialysis Tubing (Repligen, Waltham, MA, USA, 32 mm flat width, 6 kD, 1 m), SnakeSkin Dialysis Tubing (Thermo Fisher Scientific, Waltham, MA, USA, 3.5K, 35 mm dry inner diameter, 35 feet), Original Prusa i3 MK3S+Printer (Prusa Research 3D, Prague, Czech Republic), Polylactic Acid (PLA) 1.75 mm Filament (Hatchbox3D, Pomona, CA, USA), Ammonium bicarbonate (ThermoFisher Scientific, Waltham, MA, USA, 99% for analysis), Chorionic gonadotropin human (Sigma-Aldrich, St. Louis, MO, USA, 5000 IU lyophilized powder), AimStep Pregnancy Urine Cassette Test (Germaine Laboratories, Inc., San Antonio, TX, USA), Nucleocapsid Protein 95% COVID-19 (ACROBiosystems, Newark, DE, USA), Quidel QuickVue at-Home OTC COVID-19 Test (Quidel Corporation, San Diego, CA, USA), Urea (Bio Rad Lab, Hercules, CA, USA, Pkg of 1, 250 g), TCEP (Promega Corporation, Madison, WI, 15 mg), lodoacetamide (Thermo Fisher Scientific, Waltham, MA, USA), Dithiothreitol (Bio Rad Lab, Hercules, CA, USA, 1 g), Trypsin (Sigma-Aldrich, St. Louis, MO, USA, 1×Gamma-Irradiated 0.25% Porcine Trypsin 1:250 in HBSS w/0.1% EDTA-NA2 w/o CA and MG), Formic acid (Fisher Scientific, Waltham, MA, USA, 0.1% in water, Optima LC/MS, Solvent Blends), Orbitrap Exploris 480 mass spectrophotometer (Thermo Fisher Scientific, Waltham, MA, USA), EASYnLC 1200 UPLC system (Thermo Fisher Scientific, Waltham, MA, USA), Analytical column (New Objective, Inc, Woburn, MA ID 75 um) Integrafrit trap column (New Objective, Inc, Woburn, MA, ID 100 μm), ReprosilPur C18AQ 5 μm beads (Dr. Maisch, Tubigen, Germany), Formic acid (Fisher Scientific, Waltham, MA, USA, 0.1% in acetonitrile, Optima LC/MS, Solvent Blends), Water (Fisher Scientific, Waltham, MA, USA, Optima™ LC/MS Grade)

### Methods

### Water Soluble Polymers for Osmosis

Various water-soluble polymers were utilized to evaluate the rate of water movement across the membrane, driven by osmosis. The evaluation included polymers with different compositions, molecular weights, and charge properties. The polymer characteristics and the solution concentrations are summarized in Table 1:

TABLE 1
Polymers prepared at maximum mass concentration

| | Maximum mass | Molecular weight | |
|---|---|---|---|
| Polymer | concentration g/mL | (Daltons) | Charge |
| PEG 1500 | 2 | 1500 | Neutral |
| PEG 4000 | 1.5 | 4000 | Neutral |
| PEG 35000 | 0.8 | 35000 | Neutral |
| PSS | 0.6 | 1,000,000 | Negative |
| Pectin | 0.25 | Unknown | Neutral |
| PAA | 1 | 5100 | Negative |
| PEI | 1 | 25,000 | Positive |

In this study, 10 mL deionized water was loaded in the Spectra/Por 1 dialysis tubing with 32 mm flat width and 6-8 kDa MWCO. Then, the sealed dialysis bags were immersed in 80 mL of a polymer solution, listed in Table 1. After 30 minutes, the dialysis bag was removed from the polymer solution, and then briefly rinsed with DI water to remove excess polymer. The remaining water in the dialysis bag was transferred to a graduated cylinder for volume measurement. The volume difference between the initial 10 mL solution and remaining liquid was the total water removed, which was divided by the processing time (0.5 hour) to estimate the water removal rate.

### Effect of Polymer Molecular Weight on Osmosis

To evaluate the effect of polymer molecular weight on the rate of water transport across the membrane, the study utilized polyethylene glycol (PEG) at three different molecular weights, 1.5, 4, and 35 kDa. All polymer solutions of this example were prepared by pre-dissolving the polymers in deionized water at 0.8 g/mL to drive the osmosis. For the evaluation, 10 mL deionized (DI) water was loaded in the Spectra/Por 1 dialysis tubing with 32 mm flat width and 6-8 kDa MWCO. Then, the sealed dialysis bags were immersed in 80 mL of the PEG solution. After 30 minutes, the dialysis bag was removed from the polymer solution, and then briefly rinsed with DI water to remove excess polymer. The remaining water in the dialysis bag was transferred to a graduated cylinder for volume measurement. The volume difference between the initial 10 mL solution and remaining liquid was the total water removed, which was divided by the processing time (0.5 hour) to estimate the water removal rate. In one or more embodiments, one or more substances that can drive osmotic pressure can be used in addition to, or in place of, PEG.

### Effect of Polymer Solution Mass Concentration on Osmosis

To evaluate the effect of polymer solution mass concentration on the rate of water transport across the membrane, the polymer solutions were prepared using PEG 1500 (1.5 kDa PEG) to drive the osmosis. Specifically, PEG 1500 was dissolved in deionized water at concentrations of 0.125, 0.25, 0.5, 1.0, or 2.0 g/mL. For the evaluation, 10 mL deionized water was loaded in the Spectra/Por 1 dialysis tubing with 32 mm flat width and 6-8 kDa MWCO. Then, the sealed dialysis bags were immersed in 80 mL of the PEG solution. After 30 minutes, the dialysis bag was removed from the polymer solution, and then briefly rinsed with DI water to remove excess polymer. The remaining water in the dialysis bag was transferred to a graduated cylinder for volume measurement. The volume difference between the initial 10 mL solution and remaining liquid was the total water removed, which was divided by the processing time (0.5 hour) to estimate the water removal rate. In one or more alternative implementations, one or more substances that can drive osmotic pressure can be used in addition to, or in place of, PEG.

### Osmotic Processor Fabrication and Assembly

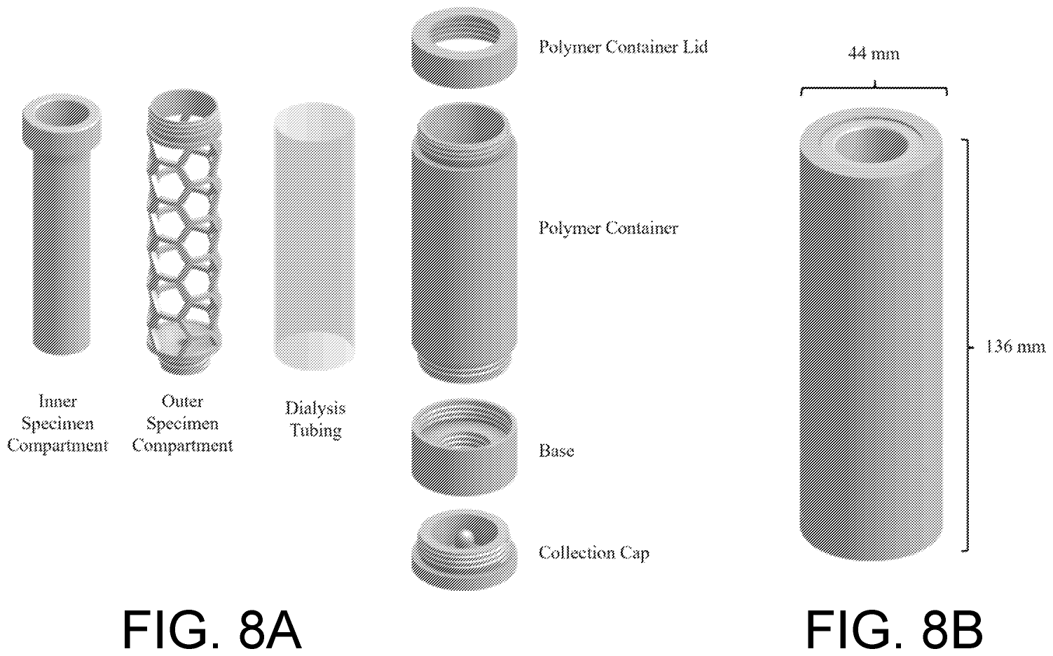

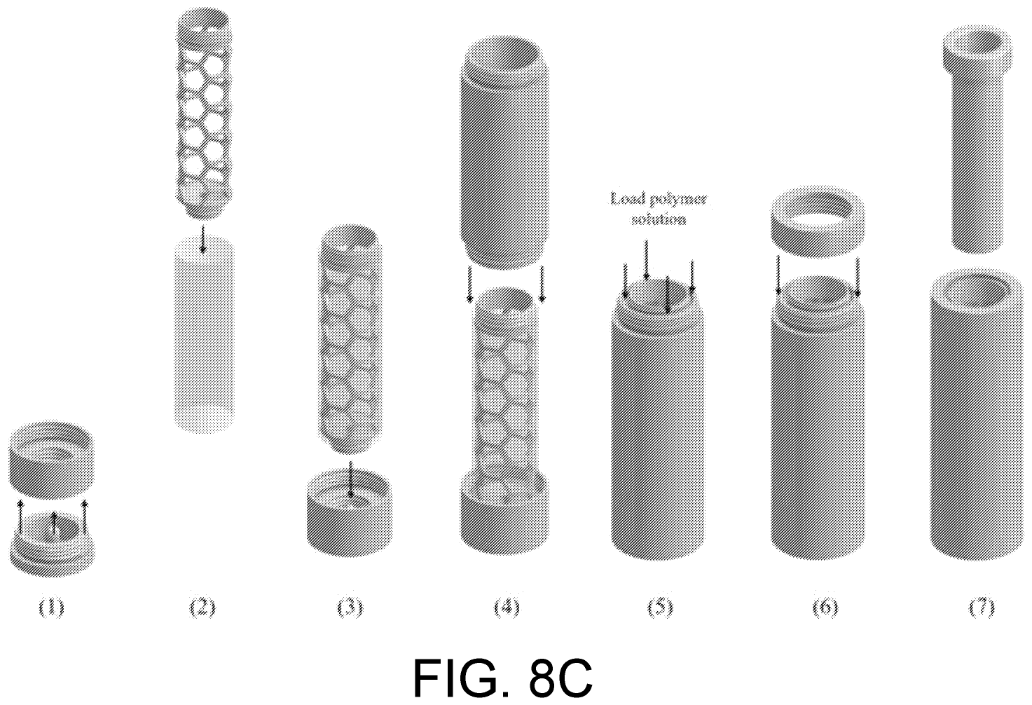

FIGS. 8A to 8C show the example device components, dimension, and assembly workflow. FIG. 8A illustrates components of the osmotic processor, including the urine compartment, polymer container lid, polymer container, base, and collection cap components. The components illustrated in FIG. 8A were fabricated using the fused filament fabrication (FFF) 3D printing method on the Original Prusa 3 MK3S+printer (Prusa Research 3D, Prague, Czech Republic) with 1.75 mm PLA filament. FIG. 8B illustrates dimensions of the assembled device. For instance, the height and diameter of the assembled device were 136 mm and 44 mm respectively. FIG. 8C illustrates the assembly workflow. First, the sample collection cap was secured to the base of the device. Next, the outer specimen compartment was inserted into 150 mm of SnakeSkin dialysis tubing with 35 mm inner diameter to hold the structure of the membrane. Next, the outer urine compartment was screwed onto the base to secure the bottom end of the dialysis tubing. Next, the polymer container was screwed onto the base. Next, the polymer container was filled with 50 mL of polymer solution. Next, the polymer container was sealed with the polymer container lid. Finally, he assembled device was ready for a fluid specimen to be added.

Concentrating Human Chorionic Gonadotropin (hCG) Hormone for Improved Lateral Flow Assay Detection Limit

hCG solutions were processed using the osmotic processor, and then the concentrated specimens were assayed using the AimStep pregnancy test, a LFA. The osmotic processor utilized 50 mL of PEG 1500 at 2 g/mL and the 6-8 kDa Spectra/Por 1 dialysis tubing. Specimens were prepared by diluting hCG in deionized water. 10 mL of the 0.02 μg/mL hCG solution was processed for 45 minutes, and the resulting 100 μL sample was assayed using the AimStep pregnancy test by following the vendor's protocol. 0 μg/mL hCG solution was also processed and assayed as a negative control. For reference, the stock solutions with 0, 0.02, and 2 μg/mL hCG were assayed using the AimStep pregnancy test. The results were recorded by capturing the images of the assays, using an Epson Perfection v39 photo scanner. The test band intensity of the hCG LFA was analyzed with ImageJ (SCHNEIDER, C. A., et al., 2012. Nat. Methods, 9, 671-675), an image-processing software, to produce semi-quantitative comparisons between the specimens before and after the osmosis. The signal was measured in arbitrary units by inverting the image color, selecting an area on the test band, and measuring the raw integrated pixel density of the selected area. For the semi-quantitative comparisons, the test line signals were further processed by normalizing against the background noise, signals from a no color membrane area away from the test and control bands.

### Concentrating SARS-CoV-2 Nucleocapsid (N) Protein for Improved Lateral Flow Assay Detection Limit

Solutions of SARS-CoV-2 N protein were processed using the osmotic processor, and then assayed with the QuickVue test, a LFA. The osmotic processor utilized 50 mL of PEG 35000 at 0.8 g/mL and the 3.5 kDa SnakeSkin dialysis tubing. Specimens were prepared by diluting the SARS-CoV-2 N protein in the 50 mM ammonium bicarbonate buffer. 10 mL of the 0.04 ng/mL N protein solution was concentrated by the osmotic processor for 1.5 hours, and the resulting 100 μL sample was assayed using the QuickVue test. 0 ng/mL N protein solution was also processed and assayed as a negative control. For reference, the stock solutions with 0, 0.04, and 4 ng/mL N protein were assayed using the QuickVue test. The results were recorded by capturing the assay membrane images, using an Epson Perfection v39 photo scanner. The test band intensity of the SARS-CoV-2 LFA was analyzed with ImageJ (Schneider et al., 2012), an image processing software, to produce semi-quantitative comparisons between the specimens before and after the osmosis process. The signal was measured in arbitrary units by inverting the image color, selecting an area on the test band, and measuring the raw integrated pixel density of the selected area. For the semi-quantitative comparisons, the test line signals were further processed by normalizing against the background noise, signals from a no color membrane area away from the test and control bands.

### Mass Spectrometry Assays for Quantitating Analyte Enrichment via the Osmosis Processor

The analyte enrichment was also characterized via mass spectrometry using SARS-CoV-2 N protein as the model analyte. To accommodate the mass spectrometry LOD, which is significantly higher than LFA, the evaluation utilized specimens with 4 μg/mL N protein. The aforementioned approach was employed to concentrate SARS-CoV-2 N protein via the osmotic processor. A calibration was generated by performing mass spec analysis using 100 μL of standard solutions with 0, 0.08, 0.4, 2, 10 and 40 μg/mL N protein.

Specimen preparation for mass spectrometry followed a published protocol (BEYNON, R. J., et al., 1989. Proteolytic enzymes: a practical approach, Oxford New York, Oxford New York: IRL Press at Oxford University Press). In brief, reduction was carried out by incubating 100 μL of each sample with 2.5 μL of 200 mM tris(2-carboxyethyl)phosphine hydrochloride (TCEP) at 37° C. for 1 hr, followed by alkylation with 20 μL of 200 mM iodoacetamide at room temperature in the dark. Excess iodoacetamide was quenched by incubation of the sample with 20 μL 200 mM dithiothreitol (DTT) at room temperature. The samples were diluted by adding 700 μL of 50 mM ammonium bicarbonate, after which tryptic digestion was carried out at 37° C. overnight. Prior to liquid chromatography/mass spectrometry (LC/MS) analysis, all samples were dried down and resuspended in 0.1% formic acid in water.

All samples of this example were analyzed on an Orbitrap Exploris 480 mass spectrophotometer equipped with an EASYnLC 1200 UPLC system and an in-house developed nano spray ionization source. Samples (5 μl at various concentrations) were loaded from the autosampler onto a 100 μm ID Integrafrit trap packed with Reprosil-Pur C18-AQ 120 Å 5 μm material to a bed length of 2.5 cm with a volume of 18 μl at a flow rate of 2.5 μL/min. After loading and desalting with 0.1% formic acid in water, the trap was brought in-line with a pulled fused-silica capillary tip (75-μm i.d.) packed with 35 cm of Reprosil-Pur C18-AQ 120 Å 5 μm. Peptides were separated using a linear gradient, from 6-45% solvent B (LCMS grade 0.1% formic acid, 80% acetonitrile in water) in 60 min at a flow rate of 300 nL/min. Peptides were detected using a targeted Parallel Reaction Monitoring (PRM) method. After the survey scan, targeted MS/MS was performed based on the inclusion list of 23 precursors (m/z, charge state) generated by Skyline (PINO, L. et al., 2020. Mass Spectrom. Rev., 39, 229-244). Precursors were isolated in the quadrupole with an isolation width of 2 m/z. Higher-energy collisional dissociation (HCD) fragmentation was applied with a normalized collision energy of 30% and resulting fragments were detected in the Orbitrap mass analyzer at 15 k resolution (at 200 m/z) with a 300% ion count (AGC) target and a maximum injection time of 22 ms. The loop count was set to ‘All’, to generate 23 fragment ion spectra per MS1 scan.

Data processing and analysis were performed using Skyline (Pino et al., 2020). The raw data were imported and R.ITFGGPSDSTGSNQNGER.S (SEQ ID NO 1), the peptide sequence, was selected for analysis due to its high abundance. The peak area under the intensity vs. retention time curve of the selected sequence was calculated by the Skyline software and correlated to the N protein standards' concentration as the calibration. Then, the peak areas of the unprocessed and processed N protein samples were used to estimate the specimen concentration via the calibration.

### Results and Discussion

### Osmosis Driven by the Polymer Solutions

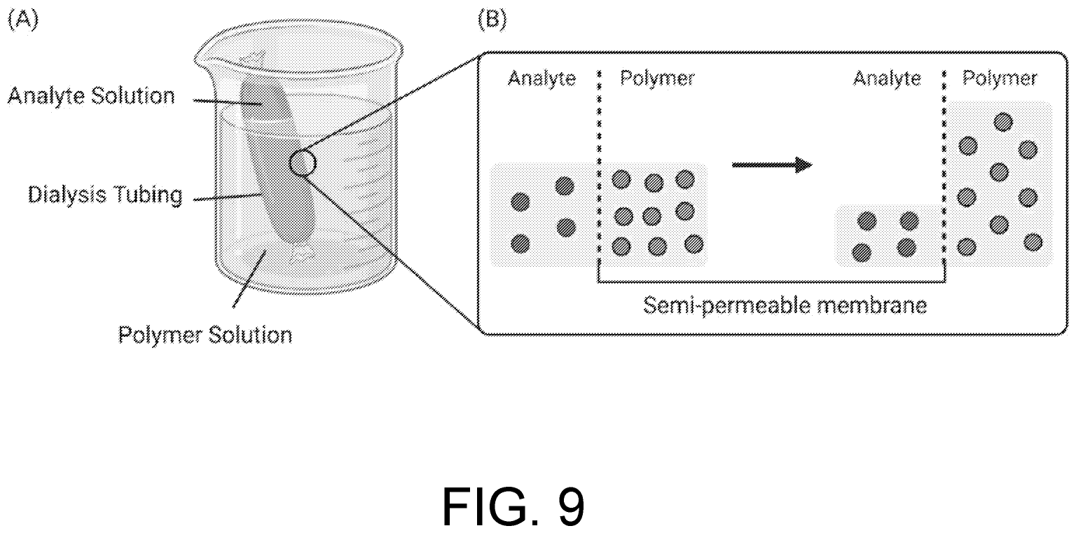

FIG. 9 illustrates a process of osmosis applied in an example urine specimen processor. Part (A) illustrates the analyte solution in a sealed dialysis tubing, which is immersed in a highly concentrated polymer solution. Part (B) illustrates that the osmotic pressure difference drives water molecules from the analyte solution across the semi-permeable membrane to the polymer solution until equilibrium has been reached.

To concentrate the urinary biomarkers, the devices of this example employed osmosis to remove water molecules from the specimens, as illustrated in FIG. 9. The beaker contains an aqueous polymer solution and a dialysis bag with the specimen inside (e.g., urine). The osmosis occurs when the dialysis bag is placed in the polymer solution due to the osmotic pressure generated by the polymer solution. The membrane MWCO is small enough to retain the analyte while allowing the transport of water and other smaller molecules. The rate of water transport across the semipermeable membrane with regard to osmotic pressure (π) (LUCKE, B., et al., 1931. J. Gen. Physiol., 14, 405-419):

$$\frac{dV}{dt} = k \times S \times ( π - {P}_{ex} ) \qquad Equation 1$$

where dV/dt is the rate of change of volume, k is the membrane permeability, S is the surface area of the membrane and Pₑₓ is the surrounding pressure. The osmotic pressure (π) produced by non-ideal polymer solutions can be described using the Flory-Huggins equation (FLORY, P. J., 1942. J. Cher. Phys., 10, 51-61; HUGGINS, M. L., 1942. Journal of physical chemistry(1952), 46, 151-158):

$$π = c \times R \times T ( {M}^{- 1} + {A}_{2} c + {A}_{3} {c}^{2} + … ) \qquad Equation 2$$

where c is the mass concentration of the polymer solution, R is the gas constant, T is the system temperature, M is the polymer molecular weight, and A2 and A3 are osmotic virial constants that describe the polymer-solvent interaction. The van′t Hoff theory is commonly applied to describe principle of osmosis in “ideal” solutions by assuming the solute and solvent particles are of similar sizes and occupy similar volumes (KENDALL, J. 1921. Journal of the American Chemical Society, 43, 1391-1396). However, the equation cannot be used here because the polymer (solute) is much larger in size and occupies more volume than the water molecules (solvent). Therefore, the Flory-Huggins equation is applied here to reflect the contribution of non-ideality to the solution osmotic pressure due to the difference in solute and solvent molecular sizes.

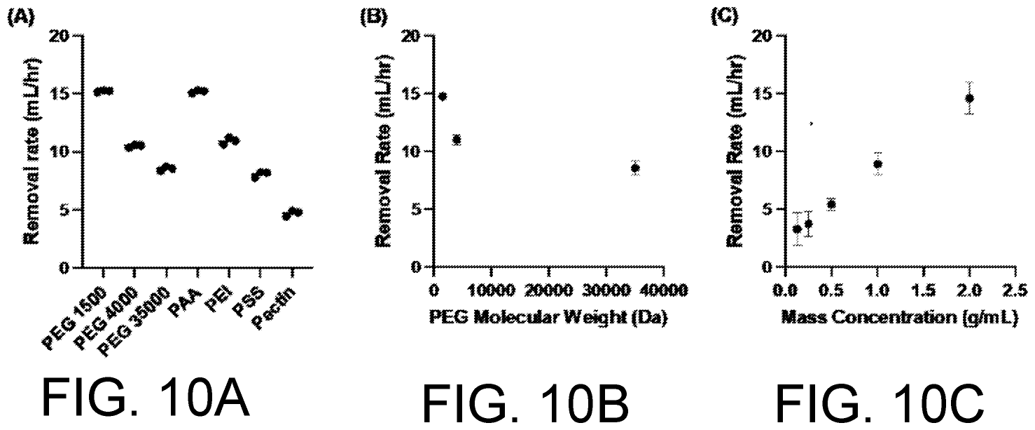

FIGS. 10A to 10C illustrate water removal rates measured in the first experimental example. FIG. 10A illustrates the removal rates associated with various polymers, including PEG 1500, PEG 4000, PEG 3500, PAA, PEI, PSS, and pectin. FIG. 10B illustrates the removal rates associated with different polymer solutions (0.8 g/mL) using PEG with 1500, 400, and 35000 Da molecular weights. FIG. 10C illustrates removal rates associated with polymer solutions using PEG 1500 with mass concentrations of 0.125, 0.25, 0.5, 1.0, and 2.0 g/mL.

The water removal via osmosis was demonstrated using various water soluble polymers (Table 1), including poly(ethylene glycol)/PEG, poly(acrylic acid)/PAAc, polyethylenimine/PEI, using the arrangement illustrated in FIG. 9. The rate of water removed from the dialysis bag varied from ca. 5 to 15 mL/hr, and was highest for PEG 1500, followed by PAA, PEI, PEG 4000, PEG 35000, PSS and Pectin (FIGS. 10A to 10C). Because lower molecular weight leads to higher water solubility, PEG 1500 was prepared at a higher mass concentration (2 mg/mL), resulting in a higher osmotic pressure (Equation 2) across the dialysis membrane to drive 15.2±0.1 mL/hr water removal. The removal rates were 10.5±0.1 and 8.6±0.2 mL/hr for PEG 4000 and PEG 35000 respectively. The stronger polymer-water interaction via charge and the low molecular weight, 5.1 kDa, allowed the PAA solution to be prepared at a higher mass concentration, 1 g/ml, resulting in 15.2±0.1 mL/hr of water removed. The removal rates were 10.9±0.3 and 8.1±0.3 mL/hr for PEI and PSS respectively. The slower water removal associated with PEI and PSS is likely caused by lower osmotic pressure, which is a function of mass concentration (Equation 2). While PEI and PSS are both charged polymers, higher molecular weights (25 kDa for PEI and 1000 kDa for PSS) limit the solubilities, which lead to lower osmotic pressures. Pectin is not charged and likely has high molecular weight given the low maximum mass concentration, with 4.7±0.3 mL/hr water removed.

According to the Flory-Huggins equation (Equation 2), the osmotic pressure is a function of polymer molecular weight. Therefore, we further evaluate the osmosis using polymer solutions prepared by PEG with different molecular weights. All polymer solutions had the same mass concentration, 0.8 g/mL. The results are summarized in FIG. 10B. As the molecular weight of PEG increases from 1.5 to 35 kDa, the rate of water removal from the dialysis tubing decreased from 14.8±0.4 to 8.6±0.6 mL/hr. The observed phenomenon aligns with the inverse relationship between polymer molecular weight and osmotic pressure (Equation 2). Higher polymer molecular weight results in lower osmotic pressure, which slows down the change in water volume over time in the dialysis tubing (Equation 1).

We also evaluated the impact of polymer solution mass concentration for osmosis. According to the Flory-Huggins equation (Equation 2), higher mass concentration leads to stronger osmotic pressure. PEG 1500 solutions at concentrations of 0.125, 0.25, 0.5, 1, and 2 g/mL were prepared for the evaluation. The results are summarized in FIG. 10C. As the polymer solution concentration increased from 0.125 to 2 g/ml, the rate of water removal from the dialysis tubing increased from 3.3±1.4 to 14.6±1.4 mL/hr. The increase in polymer mass concentration resulted in a proportional increase of osmotic pressure, increasing the rate of water transport across the membrane (Equations 1 and 2). In one or more embodiments, another biological sample can be processed in the osmotic processor, including, but not limited to, blood, serum, tissue fluid, semen, or any other biological fluid.

### Osmotic Processor Design Specifications and Workflow

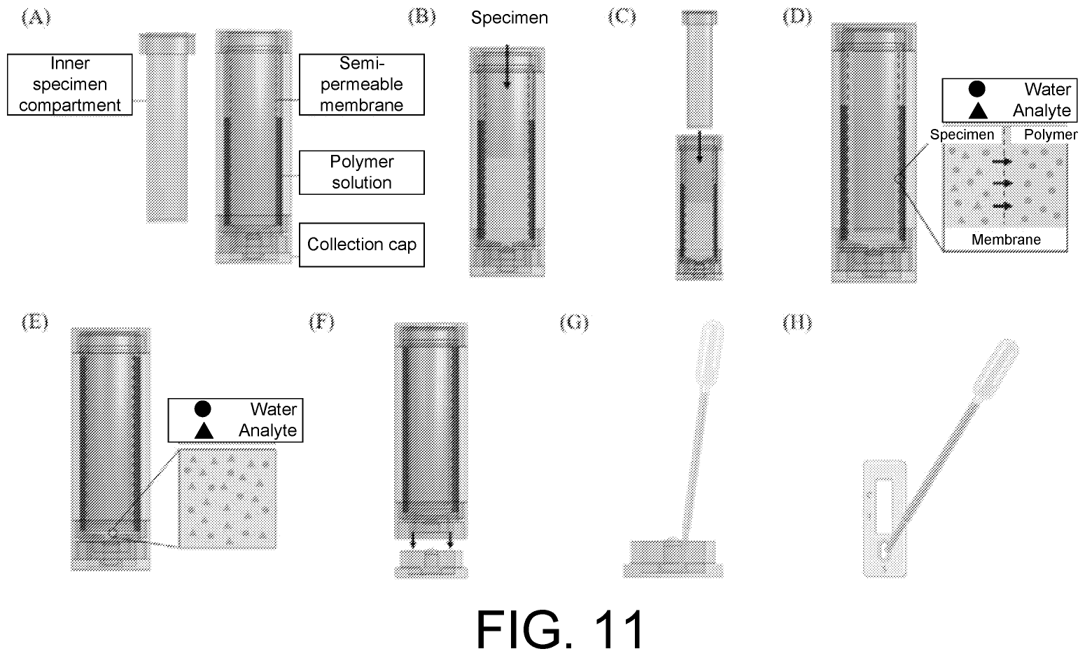

FIGS. 11A to 11H illustrate the design and workflow associated with an example osmotic specimen processing device. FIG. 11A illustrates components of the example osmotic specimen processing device. FIG. 11B illustrates that a sample is loaded into a urine compartment. FIG. 11C illustrates that an inner component of the urine compartment is inserted to create a thin specimen layer against the dialysis membrane. FIG. 11D illustrates that the processed specimen is collected at the bottom of the urine compartment with a specific volume cutoff. FIG. 11E illustrates that the specimen flows from the urine compartment to the collection cap when the collection cap is unscrewed from the remainder of the device. FIGS. 11F to 11H illustrate that the processed specimen can be pipetted from the collection cap and deposited onto a lateral flow test.

Based on our evaluation, the Flory-Huggins equation (Equation 2), and reagent availability, PEG was chosen for the osmotic processor. Assembled components of the specimen processor are shown in FIG. 11A. The workflow starts from adding the specimen to the device (FIG. 11B). Then, the inner component is insert to the urine compartment to create a thin specimen layer (FIG. 11C). To maximize membrane surface area (Equation 1), the interface between the specimen and the polymer solution, the urine specimen compartment is designed to create a thin cylindrical layer that presses against the semipermeable membrane in contact with the surrounding polymer solution. The 3.5 kDa MWCO, 35 mm dry inner diameter SnakeSkin dialysis tubing was selected to effectively retain the targeted analytes such as hCG (˜36 kDa) and the SARS-CoV-2 N protein (˜114 kDa). The polymer solution creates a pressure difference across the membrane, driving water transport from the urine compartment to the polymer container (FIG. 11D). To prevent the over-concentration and sample dry-out, a small collection cup (FIG. 11E) that has no contact with the membrane was designed at the bottom of the specimen compartment. Therefore, the final volume of the concentrated specimen is fixed at 100 μL. To collect the processed sample, the collection cup can be detached from the bottom of the device (FIG. 11F), and the sample can be deposited onto the lateral assay by pipetting (FIGS. 11G & 11H).

Concentrating Human Chorionic Gonadotropin (hCG) Hormone to Improve Lateral Flow Assay Detection Limit

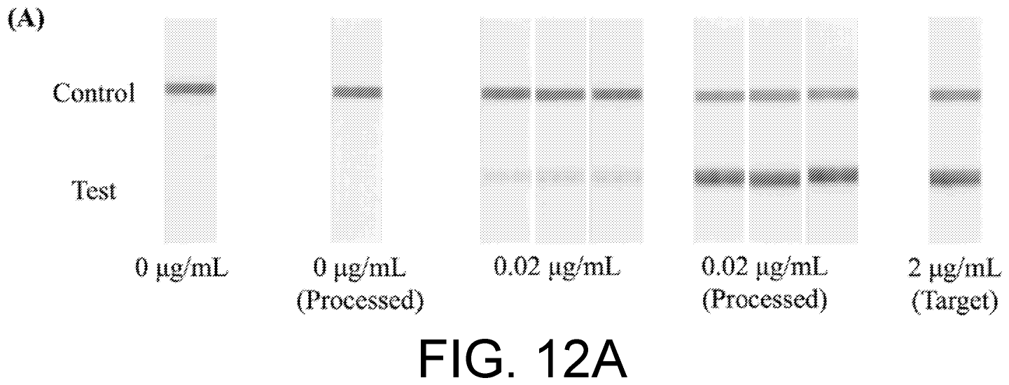

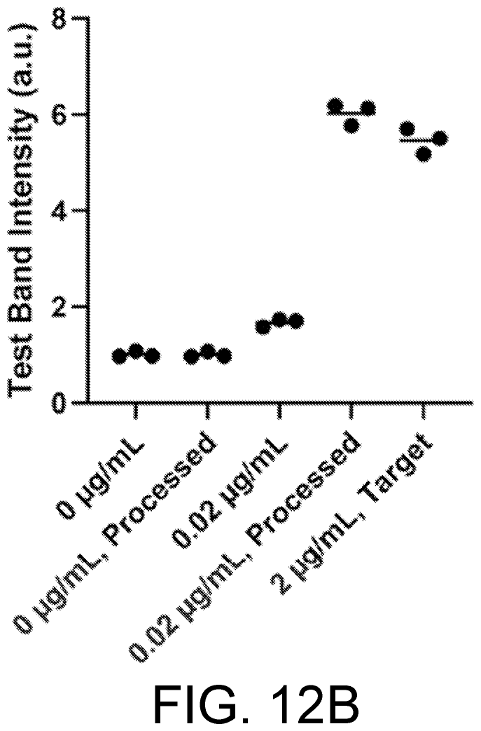

FIGS. 12A and 12B illustrate example results associated with detecting hCG hormone and an example LFA test. FIG. 12A illustrates representative images for 0 ug/mL, 0 ug/mL processed, 0.02 ug/mL, 0.02 ug/mL processed, and 2 ug/mL target hCG samples on AimStep hCG Pregnancy Lateral Flow Casettes (n=3). FIG. 12B illustrates test band intensities of blank, unprocessed, target, and processed hCG LFA tests.

To demonstrate the improved LFA detection limit, hCG hormone and AimStep® Pregnancy (an LFA) were used as a model system. The qualitative results were recorded by capturing the images of the assays (FIG. 12A), which were analyzed by quantitating the test line intensities as the assay signals (FIG. 12B). In this evaluation, assays with 0, 0.02, and 2 μg/mL hCG solutions were used as references. The device was utilized to process a 10 mL solution with 0 and 0.02 μg/mL hCG. After 45 minutes, the resulting 100 μL concentrated specimens were assayed using AimStep® Pregnancy. Assays with both 0 μg/mL and processed 0 μg/mL hCG solutions resulted in only a visible control line, which were correctly determined as negatives. Solutions with 0.02 and 2 μg/mL hCG resulted in both visible control and test lines, which were classified as positives. The 0.02 μg/mL hCG solutions led to faint test lines, and the test line intensities of the assays with 2 μg/mL hCG specimens were significantly higher (FIG. 12A). After the osmotic processing, the 0.02 μg/mL hCG specimens resulted in much stronger test line intensities, which were similar to the 2 μg/mL hCG assays. Assay signals, generated via ImageJ analysis (FIG. 12B), were 1.01±0.06, 1.68±0.08, and 5.46±0.27 for specimens with 0, 0.02, and 2 μg/mL hCG respectively. After the osmosis, the assay signals of the 0 μg/mL hCG controls was 1.01±0.06. The assay signals for the processed 0.02 μg/mL specimens increased to 6.02±0.23, which were almost the same as the assays with 2 μg/mL hCG, indicating a ca. 100-fold concentration.

### Concentrating SARS-CoV-2 Nucleocapsid Protein to Improve Lateral Flow Assay Detection Limit

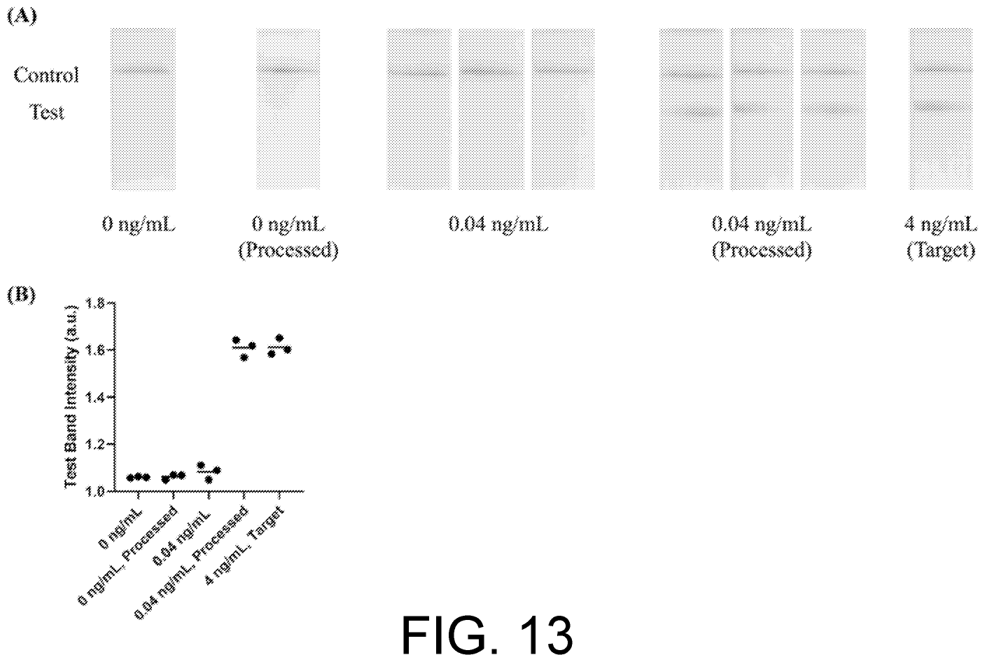

To further demonstrate the device versatility for improving LFA detection limit, SARS-CoV-2 nucleocapsid (N) protein and the QuickVue test, (an LFA) were used as a model system. FIGS. 13A and 13B illustrate example results associated with detecting SARS-CoV-2 nucleocapsid (N) protein and an example LFA test. FIG. 13A illustrates representative membrane images for 0 ng/mL, 0 ng/mL processed, 0.04 ng/mL, 0/04 ng/mL processed, and 4 ng/mL target N protein samples on Quidel QuickVue SARS-CoV-2 LFAs (n=3). FIG. 13B illustrates test band intensities of blank, unprocessed, target, and processed SARS-CoV-2 LFA tests (n=3).

The evaluation was carried out using an approach similar to the aforementioned hCG model system. The qualitative results were recorded by capturing images of the assays (FIG. 13A), which were analyzed by quantitating the test line intensities as the assay signals (FIG. 13B). In this evaluation, assays with 0, 0.04, and 4 ng/mL N protein solutions were used as references. FIG. 13A shows the assays with 0 ng/mL and processed 0 ng/mL N protein resulted in only a visible control line, which were determined as negatives. Specimens with 0.04 ng/mL N protein resulted in only a visible control line but specimens with 4 ng/mL N protein resulted in both visible control and test lines. The results indicated that the specimens with 0.04 ng/mL N protein were below the assay LOD. After the osmotic processing, the 0.04 ng/mL N protein specimens resulted in much stronger test lines, which were similar to the 4 ng/mL N protein assays. Assay signals, generated via ImageJ analysis (FIG. 13B), were 1.06±0.01, 1.08±0.03, and 1.61±0.03 for specimens with 0, 0.04, and 4 ng/mL N protein respectively. After the osmosis, the assay signal for the 0 ng/mL N protein solution was 1.06±0.01. The assay signal for the processed 0.04 ng/mL N protein specimens increased to 1.61±0.04, which was almost the same as the assays with 4 ng/mL N protein, indicating a ca. 100-fold concentration.

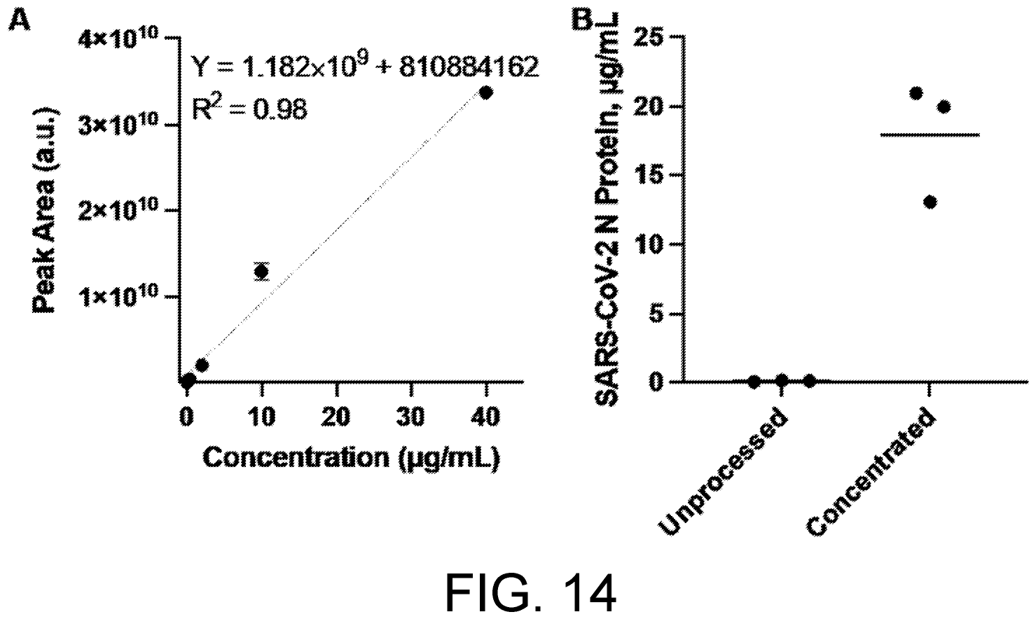

Mass spectrometry was utilized to quantitate SARS-CoV-2 N protein concentrations, which were used to estimate the enrichment factor via the osmotic processor. FIGS. 14A and 14B illustrates results of the mass spectrometry analysis used to quantify SARS-CoV-2 N protein concentration in this example. The analysis was performed by measuring the area under the ionization intensity vs. retention time peaks of the peptide sequence R.ITFGGPSDSTGSNQNGER.S. FIG. 14A illustrates a chart showing a calibration using standards, 0, 0.08, 0.4, 2, 10, and 40 ug/mL SARS-CoV-2 N protein. FIG. 14B illustrates specimens' SARS-CoV-2 N protein concentrations before and after osmosis was performed according to this experimental example. The solution average concentration was 0.09±0.06 ug/mL prior to osmosis (i.e., concentration). Then, the concentration increased to 18.0±4.3 ug/mL N protein, nearly 200-fold after enrichment.

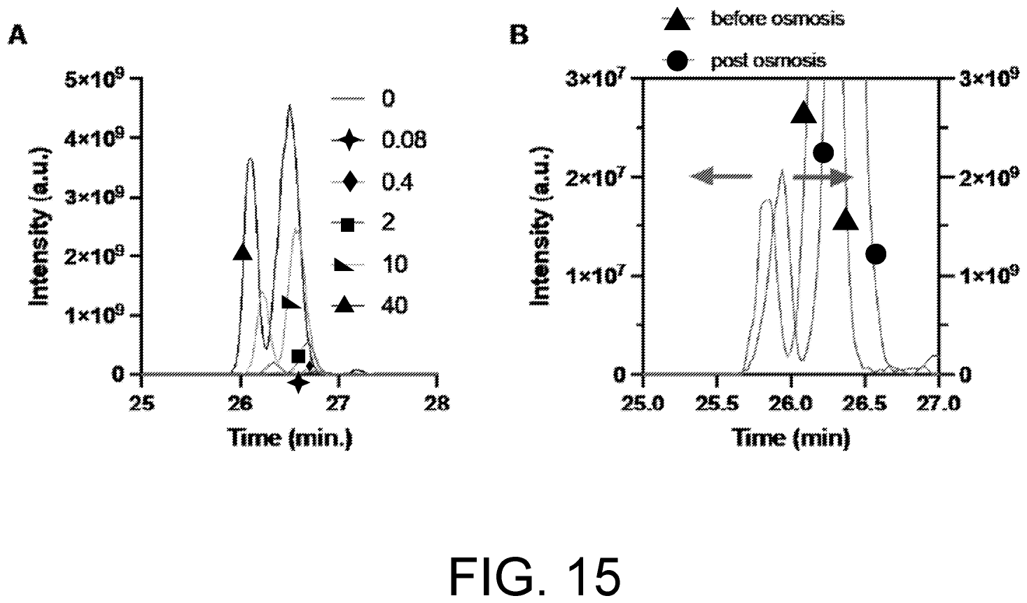

FIGS. 15A and 15B illustrate Orbitrap mass spectrometry intensity vs. retention time chromatograms of peptide sequence R.ITFGGPSDSTGSNQNGER.S (precursor 912.4114++), the peak near 26 minutes. FIG. 15A illustrates chromatograms for SARS-CoV-2 nucleocapsid protein standards 0, 0.08, 0.4, 2, 10, and 40 ug/mL. FIG. 15B illustrates chromatograms before and after the osmosis process for SARS-CoV-2 nucleocapsid protein specimens.

The enrichment factors are the concentration ratios of the processed specimens over the unprocessed solutions. To accommodate the mass spectrometry dynamic range, the evaluation utilized specimens with 4 μg/mL SARSCoV-2 N protein for the concentration process. Skyline, a software for targeted proteomics data analysis, was utilized to measure the area under the ionization intensity vs. retention time peaks of the peptide sequence R.ITFGGPSDSTGSNQNGER.S for the standards (0, 0.08, 0.4, 2, 10 and 40 μg/mL SARS-CoV-2 N protein) as well as the specimens before and after the concentration process (the peak near 26 minutes in FIGS. 15A and 15B). Because of the trypsin digestion, the specimens were diluted 10-fold prior to the mass spectrometry. The standards were utilized to generate a calibration (FIG. 14A), which was used to determine the specimen concentrations. Prior to osmosis, the specimens' average N protein concentration was 0.09±0.06 μg/mL (FIG. 14B). After the concentration process, the specimens contained 18.0±4.3 μg/mL N protein. The mass spectrometry analysis indicated that the osmotic processor concentrated the specimen nearly 200-fold. The specimen concentrations were lower than the theoretical values, which might be caused by the loss during the sample preparation.

In this study, an osmotic processor was developed to spontaneously concentrate analytes for improving the LOD of existing LFAs. The processor employed solutions with water-soluble polymers to create osmotic pressure difference across the membrane to drive the water transport, removing water from the specimen to concentrate analytes. Several polymers were evaluated and have demonstrated the capability to induce osmosis. However, the rate of water transport varies because of the polymer properties (e.g., molecular weight, charge properties). The systematic evaluations showed that faster water transport can be induced using PEG with lower molecular weights and the polymer solutions with higher mass concentration. According to the Flory-Huggins principle (Equation 2), these PEG solutions resulted in higher osmotic pressures, which drove faster water transport via osmosis (Equation 1). Therefore, PEG solutions were incorporated into the osmotic processor. To further increase the water transport rate, the specimen compartment of the processor utilizes an insert that results in a thin layer specimen solution on the membrane to maximize the contact surface area. The osmotic processor was utilized to concentrate analytes for more sensitive biomarker detections via LFAs, using hCG and SARS-CoV-2 N protein as the model analytes. After the osmosis, LFAs showed strong signals for the solutions with low analyte concentration. The analyses for the LFA membranes suggest the analyte concentrations were increased nearly 100-fold for both model analytes. Additional quantitative analyses via mass spectrometry showed the concentration of SARS-CoV-2 N protein increases ca. 200-fold. Table 2 summarizes various analyte concentration approaches, which were utilized for proteins as well as nucleic acids:

TABLE 2
Analyte enrichment by various concentration techniques

| Method | Sample type | Biomarker | Enrichment fold (ca.) |
|---|---|---|---|
| Osmotic processor (as | Ammonium bicarbonate | hCG, SARS-CoV-2 N protein | 100 |
| described in this example) | buffer | | |
| Centrifugal filtration | Urine | Circulating cathodic antigen | 100 |
| (Corstjens et al., 2015) | | | |
| Dialysis (Tang et al., 2016) | Water | Human immunodeficiency | HIV NA: 4; |
| | | virus nucleic acid (HIV NA); | MYO: 10 |
| | | myoglobin (MYO) | |
| Electrophoresis (Wu et al., | Saline sodium citrate | DNA of H5 subtype of avian | 400 |
| 2014) | | influenza virus | |
| Aqueous two-phase | PBS, FBS, synthetic urine | Transferrin | 100 |
| system (Chiu et al., 2015) | | | |
| Isotachophoresis | TE (Glycine, Bis-Tris, | Goat anti-rabbit IgG; Goat | 160-400 |
| (Moghadam et al., 2015) | pH 7.4) | anti-mouse IgG | |
| Test-zone pre-enrichment | Human blood serum; PBS | miR-210 mimic; hCG | miR-210 mimic: 10-100; |
| (Zhang et al., 2020) | | | hCG: 10 |
| Immunomagnetic | PBST; PBS | Potato virus X (PVX); 16S | PVX: 6; |
| separation (BEN AISSA, A | | rRNA gene for Myobacterium | rGM: 10 |
| et al., 2021. Sensors | | (rGM) | |
| (Basel), 21; Panferov et | | | |
| al., 2017) | | | |

These approaches concentrated analytes from few to 400-fold. Compared to the existing approaches, the osmotic processor can achieve similar enrichment and can concentrate analyte spontaneously. The osmotic processor can be utilized in conjunction with LFAs to improve biomarker detection, which can potentially increase the assay sensitivity.

### Conclusion

This example provides an osmotic processor that can spontaneously concentrate specimens' analyte to improve biomarker detections via LFA. Using hCG and SARS-CoV-2 N protein as model analytes, the osmotic processor has demonstrated the concentration process qualitatively and quantitatively. Specimens originally with analytes below the LFA LOD became detectable after the osmosis, indicating the osmotic processor concentrated the analyte to above the assay LOD. The quantitative analysis via mass spectrometry suggested 100-fold analyte concentration via the device. The device can potentially improve biomarker detection by interfacing with urine-based LFAs, many of which have low sensitivity. The analyte concentration via the osmotic processor is very comparable to other existing concentration approaches. The current design requires the transfer of enriched specimen to lateral flow test strips by manual pipetting. For further improvements in the point-of-care diagnostic workflow, the osmotic processor may be modified to seamlessly integrate with existing LFAs, where the concentrated specimen is directly released onto the sample pad of LFAs. Additionally, the device is easy to use, and does not depend on a power source, which can potentially enable many point-of-care tests (e.g., TB screening via urinary LAM) in low-resources settings.

### Second Example

Urine is a useful biospecimen that can be easily collected in large quantities with noninvasive procedures. Urine is routinely used at the point-of-care and in laboratory settings to detect pregnancies, diagnose diseases, and screen potential health problems (T. Tuuminen, Front Immunol, vol. 3, p. 45, 2012). Molecules in urine originate from glomerular filtration of plasma and excretion from and shedding of epithelial cells, representing a biomarker repertoire that can be exploited for diagnosis and monitoring of renal and systemic diseases (M. Harpole et al., Expert Rev Proteomics, vol. 13, no. 6, pp. 609-26, June 2016). Urine is composed of mostly water and solutes like urea, small ions, creatinine, albumin, bilirubin, and low concentrations of other small proteins (J. A. Simerville et al., Am Fam Physician, vol. 71, no. 6, pp. 1153-62, Mar. 15 2005; E. N. Taylor et al., Am J Kidney Dis, vol. 48, no. 6, pp. 905-15, December 2006). Concentrations of these solutes as well as the presence of other uncommon molecules are reflective of physiological conditions and can be assayed for disease diagnosis. However, diagnostic biomarkers can exist in very low concentrations well below the limits of detection (LoDs) of common diagnostic assays (S. B. Nimse et al., Analyst, vol. 141, no. 3, pp. 740-55, Feb. 7 2016). Human growth hormone (hGH), a protein biomarker for hGH secretion disorders, exists in concentrations 100-fold below available hGH immunoassays (C. Fredolini et al., Nano Res, vol. 1, no. 6, pp. 502-518, December 2008). In addition, the dilute concentration and fragmented nature of urine cell-free DNA (cfDNA), a biomarker for cancer and infectious disease diagnostics, impair the efficiency of extraction methods and consequently lower the diagnostic sensitivity of urine cfDNA detection (A. Oreskovic et al., J Mol Diagn, vol. 21, no. 6, pp. 1067-1078, November 2019). The presence of high salts and interfering molecules in urine such as biotin also hinders the development and clinical implementation of urine-based diagnostic tests (R. C. Wong et al., Lateral flow immunoassay. New York, NY: Springer, 2009, pp. xii, 223 p; R. Bowen et al., Clin Biochem, vol. 74, pp. 1-11, December 2019).

Lateral flow assays (LFAs) are immunoassays that enable paper-based in situ biomarker detection, which have been widely used in medicine, environmental health and quality control because of their low cost and rapid measurements. However, the limits of detection (LoDs) of LFAs are lower compared to laboratory-based assays. The sensitivities if LFAs are significantly affected by the low concentration of target analytes and interference (Y. Zhang et al., Sci Rep, vol. 10, no. 1, p. 9604, Jun. 15, 2020; B. Y. Moghadam et al., Anal Chem, vol. 87, no. 2, pp. 1009-17, Jan. 20, 2015). Tuberculosis (TB), the leading cause of death from a single infectious agent (Mycobacterium tuberculosis (Mtb)), can also be detected in actively infected patients via urinary LFAs (World Health Organization, “Global tuberculosis report 2020,” World Health Organization, Geneva, Switzerland, 2020). These LFAs detect lipoarabinomannan (LAM), which is a major component of the Mtb cell wall that is released during active metabolism or degradation of the bacterium and is passed via glomerular filtration into urine (M. A. Bulterys et al., J Clin Med, vol. 9, no. 1, Dec. 31, 2019). The Alere Determine™ TB-LAM Ag Lateral Flow Assay is such an LFA that has been proven to be highly specific and has potential to be a high-impact point-of-care (POC) diagnostic assay. However, this test has a poor sensitivity due to the low concentration of LAM in the urine of TB-positive patients (M. A. Bulterys et al., J Clin Med, vol. 9, no. 1, Dec. 31, 2019). LAM has on average a 14 μg/mL concentration in TB-positive patients, which is insufficient for the LAM strip test which has a limit of detection (LOD) around 500 to 1000 μg/mL (M. A. Bulterys et al., J Clin Med, vol. 9, no. 1, Dec. 31, 2019; J. I. Garcia et al., Sci Rep, vol. 9, no. 1, p. 18012, Nov. 29, 2019). Thus, this technology is not viable on its own to accurately diagnose patients.

Various techniques have been developed to improve the sensitivities of LFAs, including kinetics and transport control (L. Rivas et al., Lab Chip, vol. 14, no. 22, pp. 4406-14, Nov. 21, 2014; R. Tang et al., Sci Rep, vol. 7, no. 1, p. 1360, May 2, 2017; M. Yang et al., Virol J, vol. 10, p. 125, Apr. 22, 2013), probe- or enzyme-based signal amplification (J. Hu et al., Lab Chip, vol. 13, no. 22, pp. 4352-7, Nov. 21, 2013; C. Parolo et al., Biosens Bioelectron, vol. 40, no. 1, pp. 412-6, Feb. 15, 2013), and sample enrichment. Non-integrated enrichment techniques applied off-strip include centrifugal filtration (P. L. Corstjens et al., Parasit Vectors, vol. 8, p. 241, Apr. 22, 2015), dialysis (R. Tang et al., Talanta, vol. 152, pp. 269-76, May 15, 2016), and electrophoretic and phasic separation (J. C. Wu et al., Sensors (Basel), vol. 14, no. 3, pp. 4399-415, Mar. 5, 2014; R. Y. Chiu et al., PloS one, vol. 10, no. 11, p. e0142654, 2015), while enrichment techniques integrated on-strip include isotachophoresis (B. Y. Moghadam et al., Anal Chem, vol. 87, no. 2, pp. 1009-17, Jan. 20, 2015), aqueous two-phase systems (R. Y. Chiu et al., Lab Chip, vol. 14, no. 16, pp. 3021-8, Aug. 21, 2014), and test-zone pre-enrichment (Y. Zhang et al., Sci Rep, vol. 10, no. 1, p. 9604, Jun. 15, 2020). Although these systems have shown improved performance of LFAs, they require expensive reagents, external equipment, and/or complex procedures that are not ideal for clinical settings. On the other hand, simplified paper-based methods using dialysis or test-zone pre-enrichment have yielded suboptimal concentration folds and they have a limited processing capacity. Therefore, there is an unmet need for simplified and cost-effective methods that increase the sensitivity of LFAs.

To enable sensitive and rapid diagnosis via lateral flow assays such as the Alere TB-LAM test, this example provides an osmotic processor that concentrates urinary disease biomarkers. In the device, the passive process of osmosis drives water molecules across a semipermeable membrane where they are captured by hydrophilic polymers. The uptake of free water molecules by the polymers keeps the concentration of free water molecules low outside of the sample and thus allows osmosis to continue. The semipermeable membrane prevents large molecules from leaving while allowing small ions, solutes, and water to freely diffuse across. This traps biomacromolecules inside the membrane while removing large amounts of other molecules and ions that may interfere with the assay downstream. The feasibility for concentrating urinary analytes via osmosis has been demonstrated by McFarland, using cellulose acetate membrane and sucrose (solute) to concentrate the analytes 5-fold for gel electrophoresis (H. McFarlane, Clin Chim Acta, vol. 9, pp. 376-80, April 1964).

Compared to existing enrichment approaches, the osmotic processor demonstrates superior potential to streamline its interface with the existing point-of-care tests with its simplicity with a spontaneous process, its flexibility to process a large specimen volume, and its capability to simultaneously recondition the concentrated specimen (remove inhibitory factors) for optimal assay performance. The results described in this example demonstrate the device concept by using a model which concentrates human chorionic gonadotropin (hCG) to detectable levels and subsequently showing improved assay sensitivity. In one or more embodiments, the device can concentrate a biomolecule in a liquid solution to produce a concentrated biomolecule, such as a hormone, protein, antigen, antibody, or any other molecule. In one or more embodiments, the liquid solution can be urine, blood, water, or any liquid containing a biological sample.

### Experimental Device Design

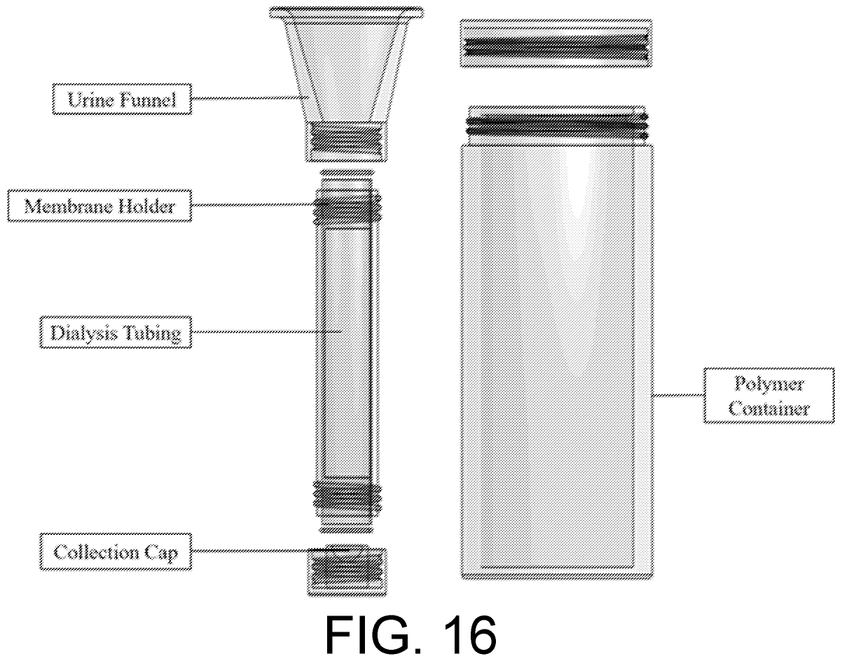

FIG. 16 illustrates the example device utilized in the second experimental example. The device includes a urine funnel, a membrane holder, a collection cap, a polymer container, dialysis tubing and polyethylene glycol (PEG) polymer. The urine funnel, membrane holder and collection cap are assembled with the dialysis tubing. The assembly is placed into the polymer container filled with PEG. The device is able to process 20-mL or any volume of urine (or biological sample) specimen, and the processed sample volume is 200-μL or approximately 1% of the specimen volume in the collection cap. While a urine funnel is described, in one or more implementations, a funnel, conical-shaped device, or any device that can facilitate flow of a fluid (e.g. urine, blood, serum, lymphatic fluid, or any other organic or bodily fluid) can be used.

### Device Fabrication

The device described in this example was fabricated using fused deposition modeling (FDM) 3D printers with polylactic acid (PLA) thermoplastic. One or more components can be fabricated from PLA or similar biocompatible polymers. In one or more implementations, polyethylene terephthalate glycol (PETG) can be used in addition to, or alternative to, PLA. 12-14 kDa, 25-mm flat width Repligen regenerated cellulose dialysis tubing is selected to retain the 36.7 kDa hCG protein that is used demonstrated the device function in this paper. 35 kDa PEG was selected as the osmotic pressure driving polymer and prepared at 0.8 g/mL. In one or more embodiments, the MWCO range could be 0.1-1000 kDa. In one more embodiments, a polymeric liquid with a selected molecular weight cut-off (MWCO) can be used in the polymer container. In an additional embodiment, the polymeric liquid can be biocompatible. In one or more embodiments, the device can be fabricated through various manufacturing methods, such as injection-molding, additive manufacturing, or other forms of 3D-printing. In one or more embodiments, a dialysis tubing can be made of any material that can facilitate osmosis.

### hCG Lateral Flow Assay

The human chorionic gonadotropin (hCG) protein is a hormone released by the human body during pregnancy. hCG LFAs are commonly used to detect the presence of hCG in urine samples for pregnancy. The limit of detection for the hCG LFA is between 2 to 10 μg/mL.

### hCG Lateral Flow Assay Calibration

hCG solution is prepared at a range of concentrations and deposited onto the LFA to establish a quantitative relationship between hCG concentration and test band intensity on the LFA. First, hCG solutions of 0, 0.005, 0.01, 0.02, 0.04, 0.08, and 2 μg/mL are prepared using deionized water. Then, 100 μL of hCG solution at each concentration are deposited on LFAs and the test band intensities of LFA are quantified with ImageJ software.

### hCG Protein Spike-and-Recovery on Device

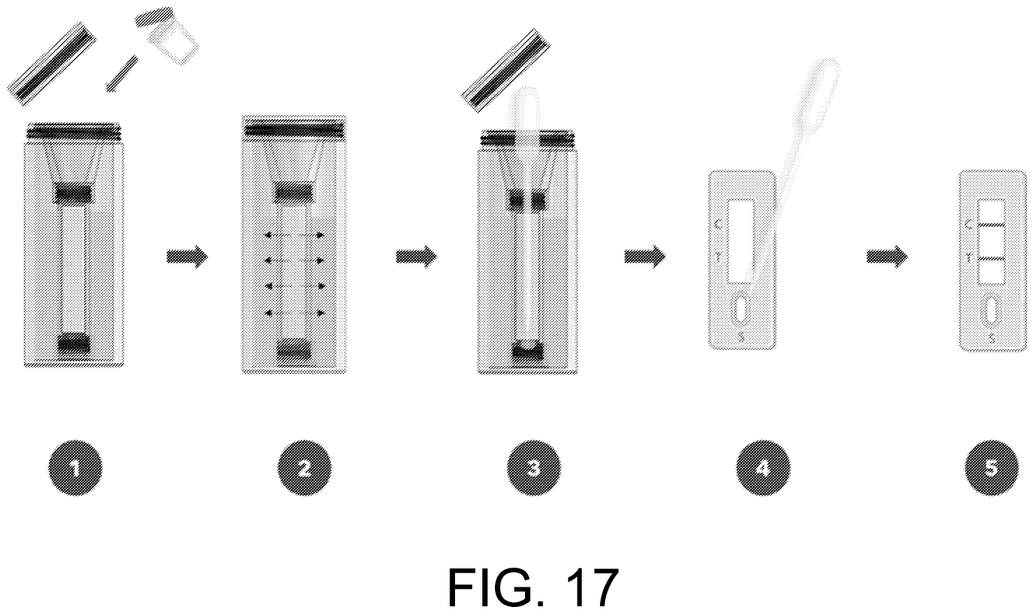

FIG. 17 illustrates the workflow of an example device. (1) Pour 25 mL of urine sample into the device and close the polymer container lid to prevent spillage. (2) The osmosis process takes place for 3.5 hours and the 25 mL urine sample is concentrated 100-fold to 250 μL of sample. (3) Remove the polymer container lid and extract the concentrate from the collection cap. (4) Deposit the concentrate on the LFA and wait for 30 minutes. (5) Read the test result.

Since the device is designed to concentrate 20 mL of urine sample 100-fold, the processing capability of the device is evaluated by concentrating 0.02 μg/mL of hCG solution. In one or more embodiments, the maximum concentration fold is 100×. Following the workflow shown in FIG. 17, 20-mL of 0.02 μg/mL hCG solution is added to the urine processor. The processing is performed near standard temperature and pressure. After 3.5 hours, 200-uL of the processed sample is pipetted from the collection cap and deposited onto the LFA. Alongside the processed sample, 0 ug/mL, 0.02 ug/mL and 2 ug/mL hCG are also prepared for qualitative comparison and quantitative calibration against the trend established in the hCG Lateral Flow Calibration procedure.

Water Removal using Polyethylene Glycol at Different Molecular Weights

PEG with 1500, 4000 and 35000 Da molecular weights were used to remove 10 mL of water from a dialysis bag. With different levels of solubility, PEG was prepared at maximum concentration at a volume of 100 mL using deionized water. The prepared PEG solutions and corresponding concentrations are listed in Table 3. 12000-14000 Da dialysis bags with 16 mm diameter are filled with 10 mL of deionized water and immersed in 100 mL of polyethylene glycol. After one hour, the water remaining in the dialysis tubing was extracted and measured to evaluate the water removal speed of PEG with different molecular weights. In one or more embodiments, the range of molecular weights for PEG was 1-35 kDa.

TABLE 3
Concentrations of polyethylene glycol
at different molecular weights.

| Polyethylene Glycol Molecular Weight (Da) | Concentration (g/mL) |
|---|---|
| 1500 | 2 |
| 4000 | 1.5 |
| 35000 | 0.8 |

### Results and Discussion

Human chorionic gonadotropin (hCG) LFA was used as a model assay to demonstrate the processing function of the device. Commercial hCG home pregnancy LFAs are readily available and have demonstrated sensitivity greater than 99% and specificity of 99.2%, making the hCG LFA an ideal model for evaluating biomarker detection at various concentrations (R. E. O'Connor et al., Am J Emerg Med, vol. 11, no. 4, pp. 434-6, July 1993).

### hCG Lateral Flow Assay Calibration

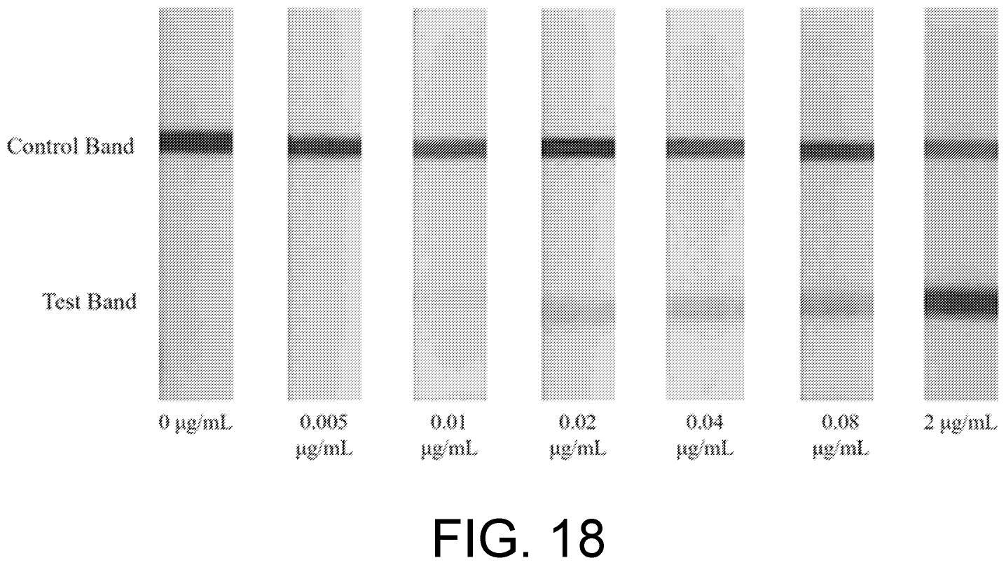

FIG. 18 illustrates lateral flow assay results for 0, 0.005, 0.01, 0.02, 0.04, 0.08, and 2 μg/mL hCG solutions for this example. hCG solution was prepared at a range of concentrations and deposited on LFA tests to generate a test band intensity vs concentration calibration trendline. hCG solutions of 0, 0.005, 0.01, 0.02, 0.04, 0.08, and 2 μg/mL generated a range of test band intensities shown in FIG. 18. Images of the LFAs are captured with a smartphone camera. The ImageJ software was used to measure the integrated pixel intensity in a selected area of the LFA test band. The test band pixel intensity was then subtracted from the pixel intensity of a blank section on the LFA strip. Afterward, the LFA test bandintensity vs. hCG concentration data was fitted with a logarithmic function to produce the calibration fit line shown by Equation 3 below:

$$Test Band Intensity = 0.1883 ln ( concentration ) + 2.083 \qquad Equation 3$$

### hCG Protein Spike-and-Recovery

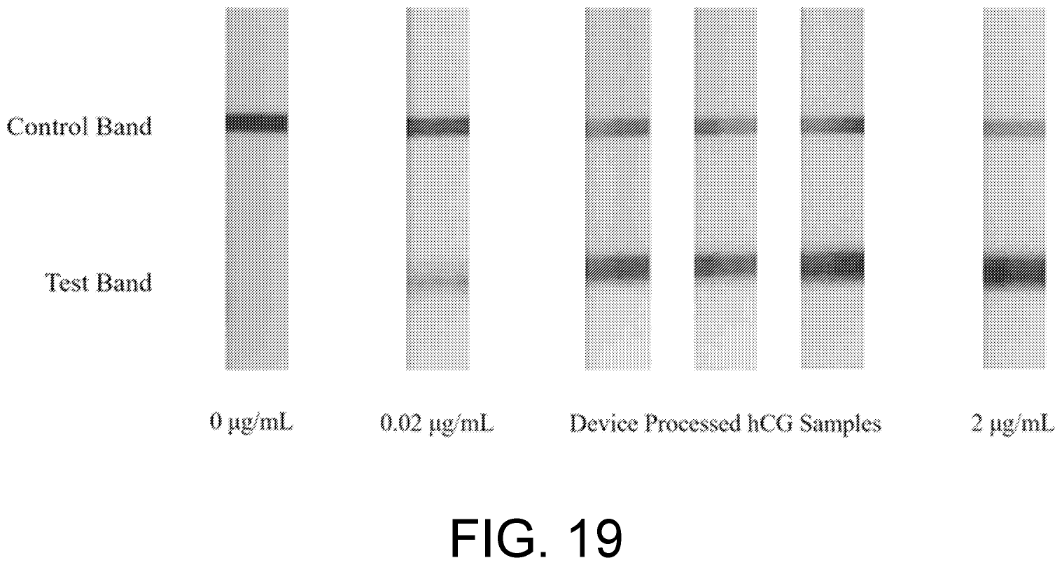

FIG. 19 illustrates results of examples of 0 μg/mL, 0.02 μg/mL (unprocessed), device-processed, and 2 μg/mL hCG LFAs. hCG protein spike-and-recovery was performed on the urine processor to demonstrate the function of the device and its ability to improve LFA test sensitivity. The device processed hCG samples, along with 0 μg/mL, 0.02 μg/mL and 2 μg/mL hCG solutions were evaluated using hCG LFAs as shown in FIG. 19. As expected, the 0 μg/mL sample displayed a blank test band, the unprocessed 0.02 μg/mL sample displayed a faint test band, while the device-processed samples displayed much darker test bands that are comparable to the test band of the 2 μg/mL target.

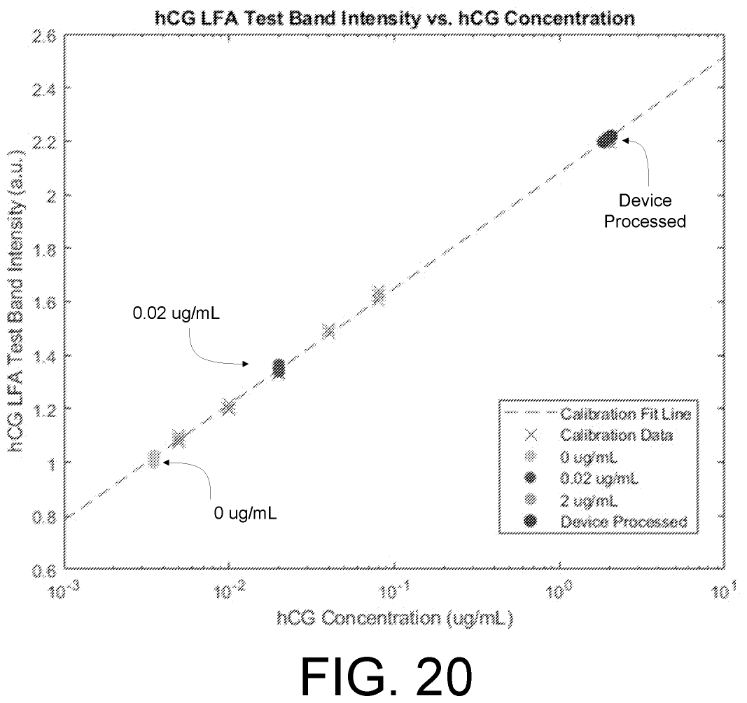

FIG. 20 illustrates hCG LFA test band intensity vs. log hCG concentration plot of 0 ug/mL, 0.02 ug/mL (unprocessed), 2 ug/mL, and device-processed data plotted along the calibration trendline in accordance with his example. Similar to the LFA calibration procedure, the LFA results were captured using a smartphone camera and the test band intensity was evaluated using ImageJ software. Based on differences between test band intensity readings of the 0 ug/mL, 0.02 ug/mL, and 2 ug/mL hCG solutions and the previously established calibration data, the test band intensity of the processed sample was scaled to fit the calibration trendline, shown in FIG. 20.

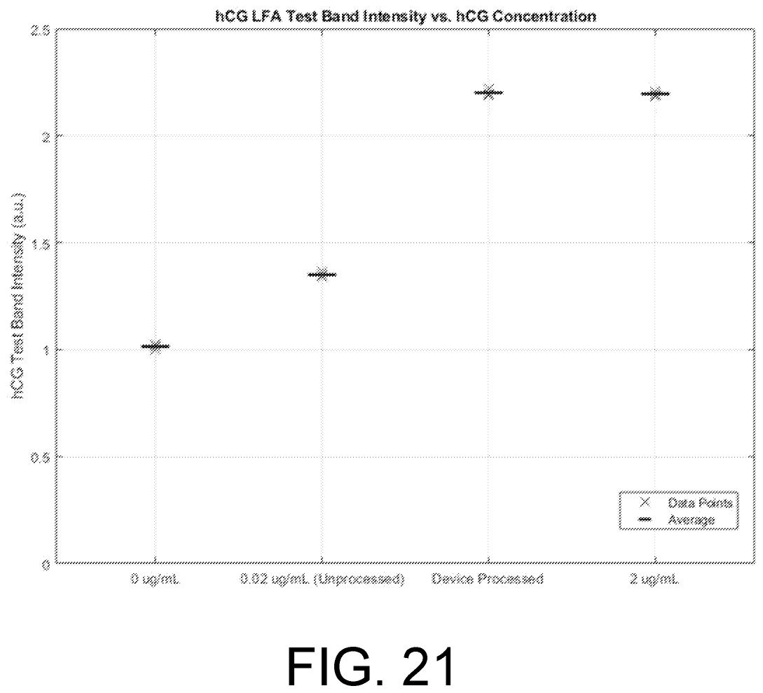

FIG. 21 illustrates test band intensity vs. hCG concentration plot of 0 μg/mL, 0.02 μg/mL (unprocessed), 2 μg/mL hCG, and processed hCG samples in accordance with this example. The scaled test band intensities of the processed sample were substituted into Equation 3 to back-calculate the corresponding concentrations of the processed sample. The concentration of the device processed samples was estimated to be 1.84 ug/mL, 2.05 ug/mL, and 1.90 ug/mL, which correlates to 92-, 102.5-, and 95-fold concentration, respectively. The results of the sample processing are summarized in FIG. 21. In one or more embodiments, the analyte concentration can be 0.01-0.1×below the target concentration of the analyte.

As observed, there was variation in concentration fold among the three trials. The 92- and 95-fold concentrations may have been caused by sample loss along the membrane or flow into connecting sections of the device. Since the regenerated cellulose membrane is already one of the commercially available membranes with the lowest non-specific protein adherence, sample loss may be addressed by further improvements in the device design. The 102.2-fold over-concentration may have been due to the compression of the membrane during osmosis that caused the polymer to further contact the solution in the collection cap. This may also have been addressed by design changes to minimize membrane contraction during device processing.

### Principle of Osmosis

While the hCG concentration experiment demonstrated near 100-fold concentration and noticeable improvement on the LFA test band visibility, the device is still not ideal for point-of-care settings due to the 3.5 hour processing time. One way to reduce the processing time includes increasing the volumetric flow rate of water across the dialysis membrane. The volumetric flow rate of water across the semipermeable membrane can be modeled by Equation 4:

$$\frac{dV}{dt} = k \cdot S \cdot ( π - {π}_{ex} ) \qquad Equation 4$$

where k is the membrane permeability, S is the membrane surface area, πₑₓ is the pressure outside of the membrane, and π is the osmotic pressure of the polymer solution (B. Lucke et al., J Gen Physiol, vol. 14, no. 3, pp. 405-19, Jan. 20, 1931). The membrane permeability is limited by the size of the target analyte and the volume to membrane surface area ratio has been set by the design process. As a result, the upcoming prototypes aim to increase the osmotic pressure of the polymer solution. The virial expansion of the osmotic pressure, π, is given by:

$$π = c \cdot R \cdot T ( \frac{1}{M} + {A}_{2} c + {A}_{3} {c}^{2} + … ) \qquad Equation 5$$

where c is the polymer molar concentration, R is the ideal gas constant, T is the temperature, M is the molecular weight of the polymer, and A₂ and A₃ are the second and third virial coefficients associated with solute-solvent interactions (P. J. Flory, Principles of polymer chemistry (The George Fisher Baker non-resident lectureship in chemistry at Cornell University). Ithaca, N.Y.: Cornell University Press, 1953, p. 672). Manipulating the temperature is not ideal for low-resource, point-of-care settings as it would require an external power source. By reducing the molecular weight and increasing the concentration of the polymer, the osmotic pressure may be increased to raise the volumetric flow rate of water across the membrane.
   Water Removal using Polyethylene Glycol at Different Molecular Weights

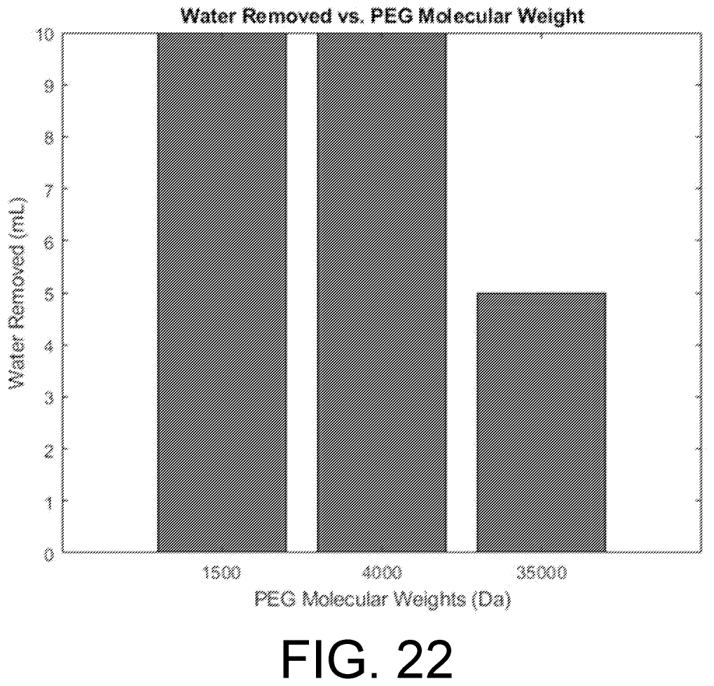

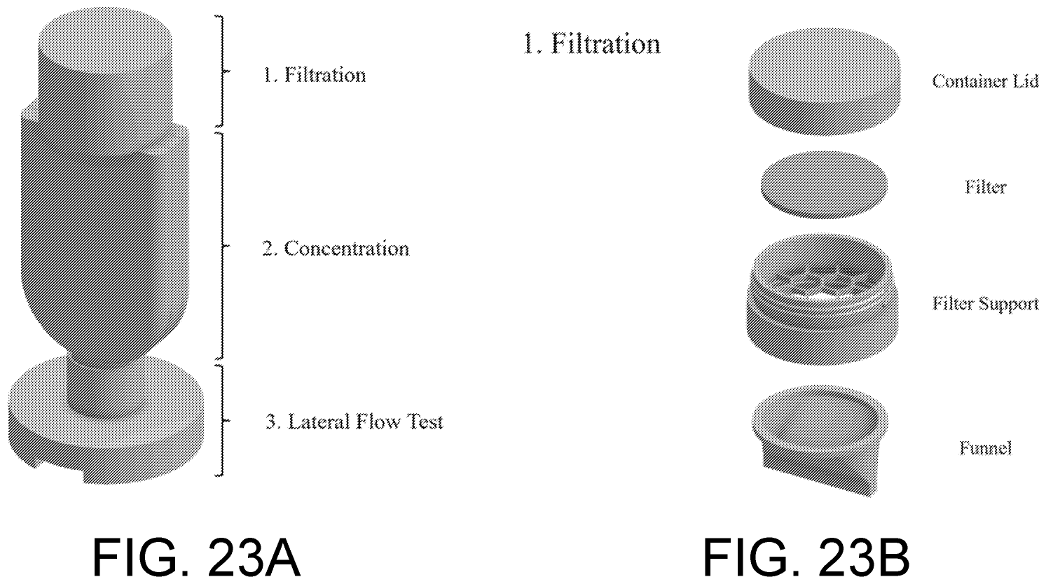

FIG. 22 illustrates an example of water removed vs. PEG molecular weight (prepared at maximum concentrations). To improve the 3.5 hour processing time, PEGs with molecular weight smaller than 35000 kDa are tested to remove water from the dialysis tubing. According to Equation 5, by increasing the concentration of the solution on one side of the membrane increases the osmotic pressure across the membrane. The increase in osmotic pressure across the membrane increases the water flow rate across the membrane, as described by Equation 4. The PEG 35000 used to demonstrate hCG spike-and-recovery was prepared at the highest concentration possible. To further increase the concentration of polymer used on the urine processor, PEGs with lower molecular weight and higher solubility were prepared. 100 mL of PEG 1500, 4000, 35000 at concentrations of 2, 1.5, and 0.8 g/mL, respectively, remove 10 mL of water in a dialysis bag in 1 hour. PEG 1500 and 4000 were able to remove 10 mL within 1 hour, while PEG 35000 was only able to remove 5 mL, as shown in FIG. 22. The difference in water removal rate demonstrates potential for faster concentration on the urine processor.

### Conclusions

In A novel osmotic concentrator that achieves near 100-fold concentration of hCG without a power supply is described herein. Future studies will focus on optimizing the device design to precisely control the final sample concentration, as well as decreasing the suboptimal processing time by using affordable polymers with high solubility. Future work will confirm the device's utility in concentrating LAM in urine to increase the sensitivity of the Alere TB-LAM test. Ultimately, this will allow the Alere TB-LAM test to meet the WHO TPP as a POC TB test that overcomes the limitations of currently available diagnostic tools. Along with the Alere TB-LAM test, the osmotic concentrator is expected to improve the diagnostic rate of TB in low-resource settings for early disease control and a reduction in mortality. Because of the adaptable nature of the concentration mechanism, the device also shows a great potential to be used in concentrating other disease biomarkers including DNA, proteins, and exosomes for diagnostic, disease monitoring and therapeutic purposes.

### Third Example

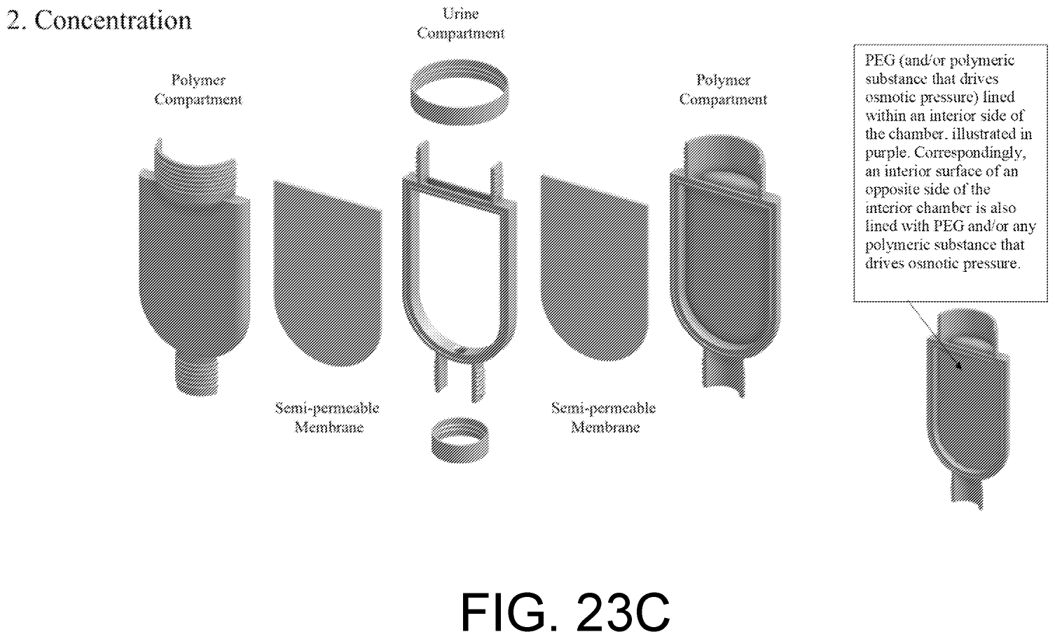

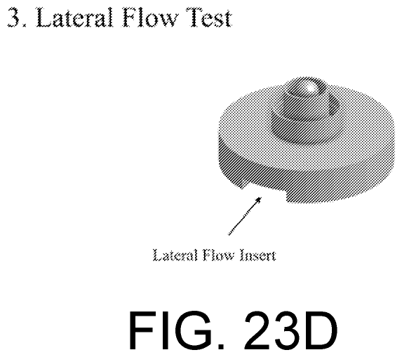

FIGS. 23A to 23D illustrate an example device for concentrating and collecting biomolecules via osmosis. FIG. 23A illustrates overall components of the device. FIG. 23B illustrates filtration components of the device. FIG. 23C illustrates concentration components of the device. FIG. 23D illustrates a collection component of the device that is configured to connect to a lateral flow test.

In various implementations, the device can include a filtration zone, a concentration zone, and a lateral flow test zone. In the filtration zone, an embodiment can comprise a filter membrane that is seated in a support chamber. The filter membrane can be a filter paper, a nitrocellulose membrane, polyethersulfone membrane, nylon, or similar material. In one or more embodiments, the support chamber can have a grid in different geometrical shapes (e.g. hexagonal, square, circular, etc.). Coupled to below the support chamber is a fluid collection channel. In one or more embodiments, the fluid collection channel can be a funnel, cone, or a channel that facilitates holding and channeling the fluid to a subsequent container. In additional implementations, the filtration component can have a lid to seal the filtration component and device. A fluid sample (such as urine or any fluid for testing) is poured into the filtration component, where the filter membrane removes unwanted debris from the sample, and facilitates transport of the sample downstream for further analysis.

In various implementations, the concentration zone can comprise a chamber. In an embodiment, the chamber can comprise a cage with a groove outlined around the perimeter of the cage. In an additional implementation, an elastomeric seal is placed inside the outline of the groove to seal the chamber. The elastomeric seal can be made of rubber, silicon, polyurethane, or similar material. Seated in and/or around the groove is a semi-permeable membrane that is oriented in a direction parallel to the cage. While the illustration presents the semi-permeable membrane is a half-oval shape, it is foreseeable that other geometries (half/whole circle, rectangle, square, triangle, etc.) can be used in the device and not have its function affected. In one or more embodiments, the semi-permeable membranes are spaced apart in a predetermined distance to facilitate containment of the fluid sample in between the semi-permeable membranes. In one or more implementations of the chamber, PEG, and/or any polymeric substance that drives osmotic pressure, is lined across a portion of an interior surface of the chamber in close proximity of the semi-permeable membrane. Correspondingly, an interior surface of the opposite side of the chamber is lined with PEG and/or any polymeric substance that drives osmotic pressure. A fluid sample disposed in the filtration zone travels down to the concentration zone, where the fluid sample is contained between the semi-permeable membranes. As the selected polymeric substance drives osmotic pressure within the chamber, the solvent of the fluid sample is pulled through the semi-permeable membranes and out of the space between the semi-permeable membranes, leaving a concentrated fluid sample that contains a biomolecule for testing. The filtration zone and the concentration zone are removably coupled via a screwing connection, though other forms can be utilized that create a seal that prevents fluid from leaking outside of the overall device (e.g. ratcheting, slotted, peg, dovetail, etc.).

In an embodiment, the lateral flow test zone can comprise a slot dimensioned to receive a lateral flow insert, whether it is a testing device or a testing strip. In an additional embodiment, the lateral flow test zone can include a collection channel that transports the concentrated fluid sample to the lateral flow insert. In one or more implementations, the collection channel can be dimensioned to receive and send a desired amount of the concentrated fluid sample to the lateral flow insert, depending on how much of the concentrated fluid sample is necessary for testing. In one or more embodiments, the volume of the collection channel can range from 0.01-0.1× the starting sample volume, depending on the sample volume and the target concentrate volume. Parameters of the device can be adjusted in accordance with the sample volume and target concentrate volume. In one or more embodiments, the concentrated fluid sample can comprise one or more biomolecules that can correspond to the testing parameters of the lateral flow insert. In one or more cases, the biomolecule can be collected for diagnosing ailments, screening for maladies, or testing efficacies of pharmaceutical drugs.

### Fourth Example

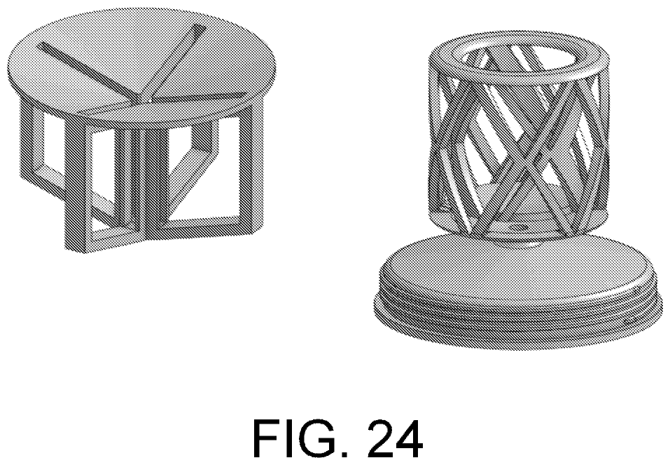

FIG. 24 illustrates an example concentrator housing.

### Fifth Example

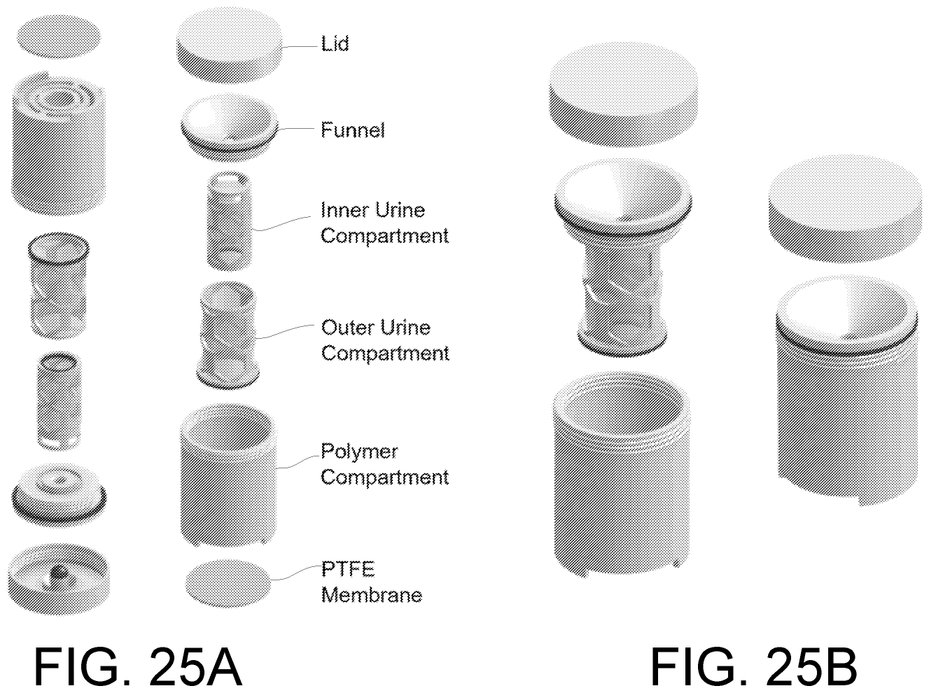

FIGS. 25A and 25B illustrate components of an example concentrator. This example concentrator includes two membranes (held by respective membrane holders), one disposed between a sample chamber and an inner volume, and another disposed between the sample chamber and an outer volume. The sample chamber, for example, is configured to receive and hold urine. For instance, the sample chamber of the concentrator illustrated in FIGS. 25A and 25B can be referred to as a urine compartment. A polymer solution is disposed in the inner volume and the outer volume. Thus, during operation, a solvent (e.g., water) flows into the solution disposed in the inner volume and the outer volume.

In addition, the example concentrator illustrated in FIGS. 25A and 25B includes a funnel that is configured to be coupled to a sample collector after the urine is loaded into the sample chamber. Thus, the inlet of the sample chamber is also the outlet of the sample chamber, such that the urine is loaded and removed from the sample chamber at the same end.

Further, the sample chamber of the example concentrator illustrated in FIGS. 25A and 25B is vented. In particular, a PTFE membrane is disposed in a channel that extends from the sample chamber to an exterior of the concentrator (e.g., the atmosphere). Air may pass through the PTFE membrane into the sample chamber as water is drawn from the sample chamber into the volumes of the polymer solution.

FIG. 25A illustrates various components of the example concentrator. Specifically, the left side of FIG. 25A illustrates the components arranged such that the lid is pointed downward, and the right side of FIG. 25A illustrates the components such that the lid is pointed upward. FIG. 25B illustrates the components partially assembled into the concentrator. For instance, the left side of FIG. 25B illustrates the funnel, inner urine compartment, and outer urine compartment assembled together, as well as illustrates the PTFE membrane inserted into the polymer compartment. The right side of FIG. 25B illustrates funnel-inner urine compartment-outer urine compartment assembly inserted into the polymer compartment.

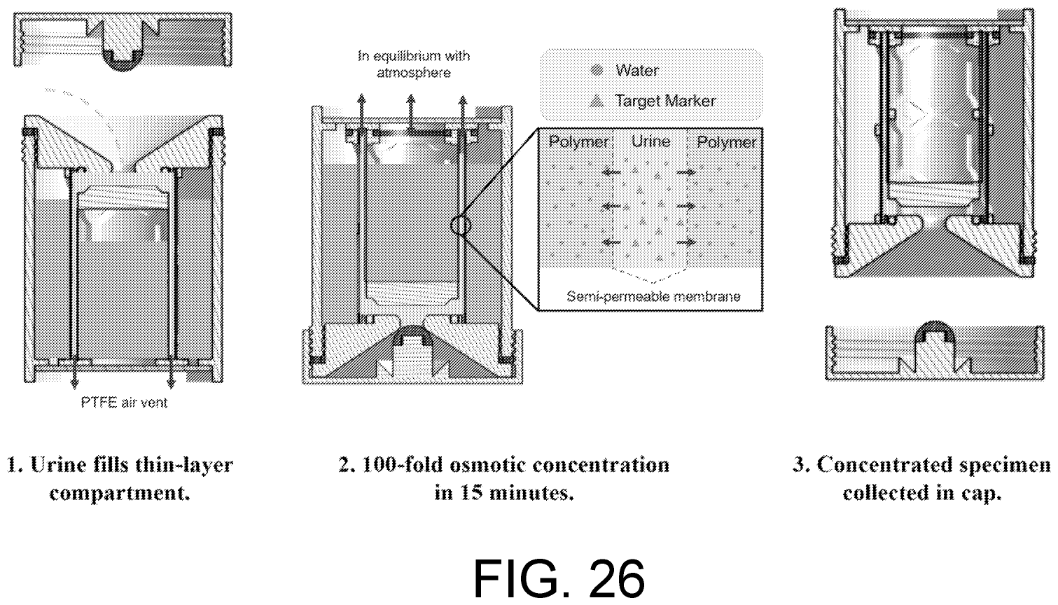

FIG. 26 illustrates an example of the interior operation of the concentrator in the Fifth Example. First, urine is loaded into a sample chamber (i.e., a compartment). Due to the relatively short distance between the inner and outer membranes, the urine is disposed in a thin layer between the inner and outer membranes. Second, the two volumes of the polymer solution draw water out of the urine sample through the two membranes. In preliminary experimental data, it was observed that this device could achieve 100-fold osmotic concentration of the urine in 15 minutes. Third, the concentrated urine specimen is collected in the cap. The concentrated urine specimen, for example, can be tested for an analyte-of-interest using a diagnostic assay.

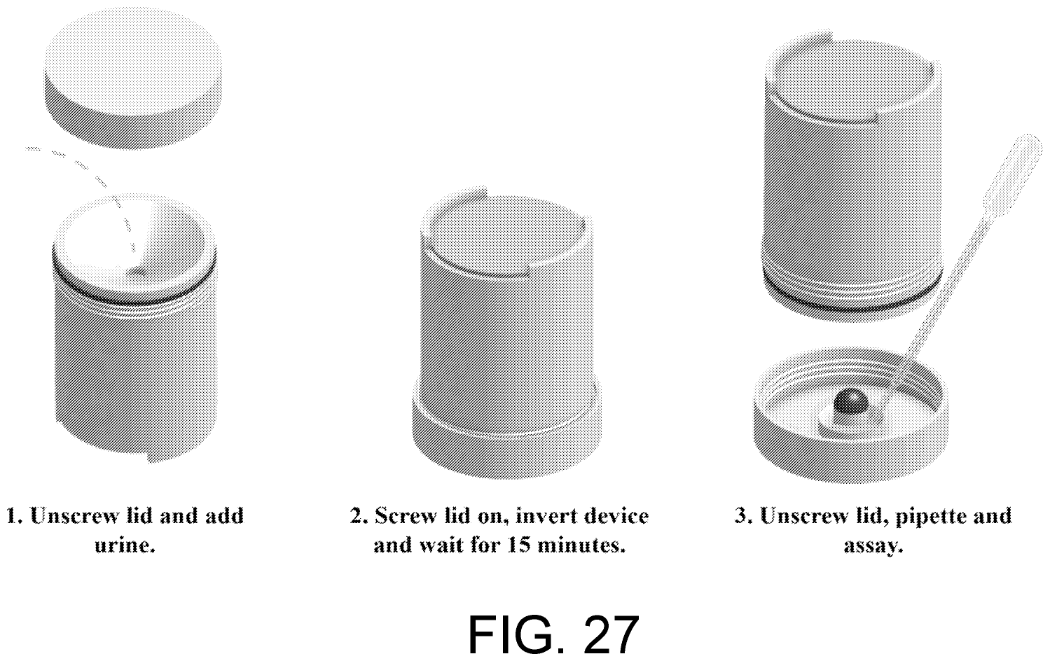

FIG. 27 illustrates an example of the exterior operation of the concentrator in the Fifth Example. First, a user may unscrew a packaging lid from the concentrator and load the concentrator with a urine sample using a funnel. Second, the lid may be screwed back onto the concentrator. The user may invert the concentrator as the urine specimen is concentrated. For instance, the user may wait for 15 minutes. Third, the user may unscrew the lid, which may yield a predetermined volume of a concentrated urine sample. The user may use a pipet to move the predetermined volume from the lid for further analyte detection.

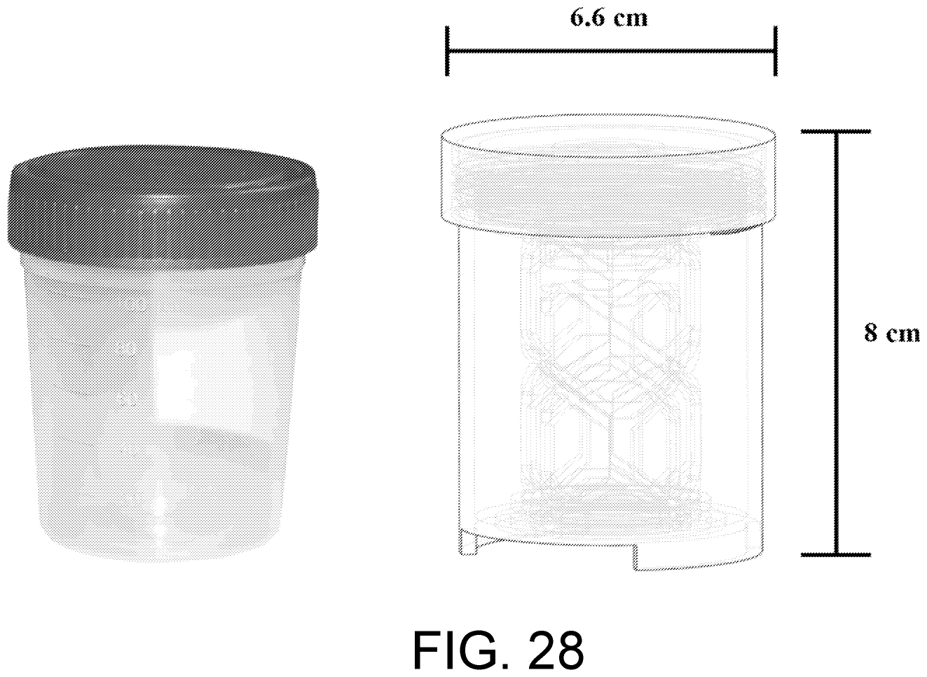

FIG. 28 illustrates example dimensions of the concentrator in the Fifth Example. As illustrated, the concentrator (right) may have the approximate dimensions of a standard urine specimen cup (left). In particular cases, the concentrator has a diameter of 6.6 cm and a height of 8 cm. However, implementations of the example concentrator in the Fifth Example are not limited to the specific dimensions illustrated in FIG. 28.

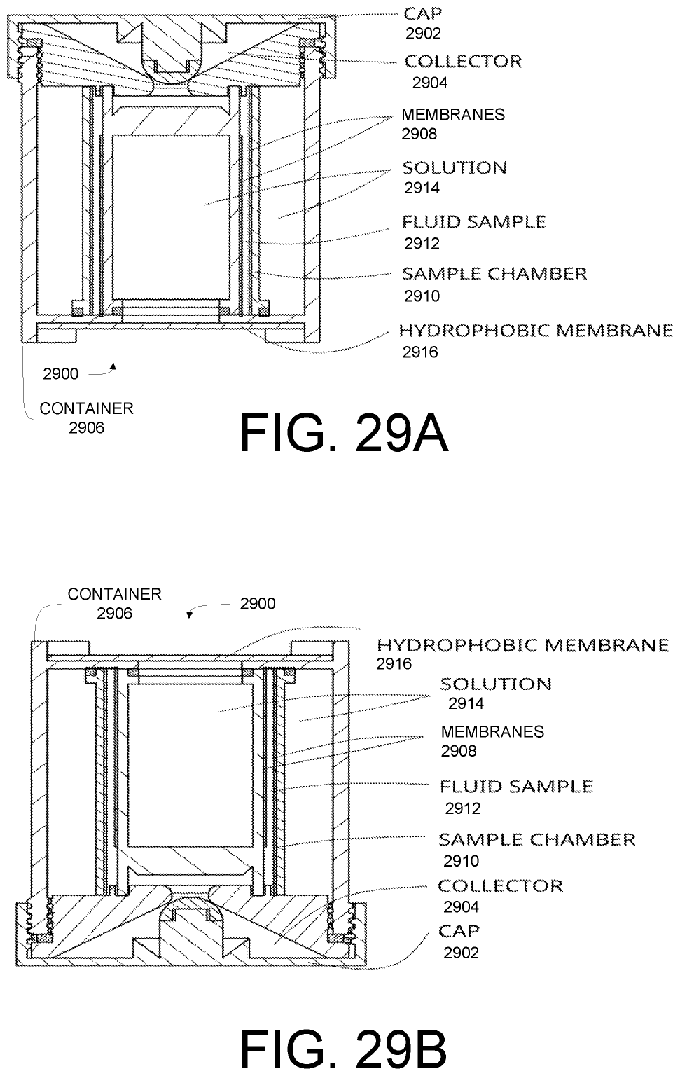

FIGS. 29A and 29B illustrate diagrams of an example concentrator 2900 of the Fifth Example. FIG. 29A illustrates the concentrator 2900 in a first orientation. FIG. 29B illustrates the concentrator 2900 in a second orientation. Specifically, the concentrator 2900 includes a cap 2902 that is removably connected to a collector 2904 (e.g., a funnel) and a container 2906, which serves as an outer housing for the concentrator 2900. Two membranes 2908 are disposed in an interior space of the container 2906. A sample chamber 2910 is defined between the two membranes 2908. A fluid sample 2912 is disposed in the sample chamber 2910. In addition, two volumes of a solution 2914 are disposed inside of the concentrator 2900. One of the membranes 2908 is disposed between the fluid sample 2912 and a first volume of the solution 2914, and the other one of the membranes 2908 is disposed between the fluid sample 2912 and a second volume of the solution 2914. The concentrator 2900 further includes a hydrophobic membrane 2916, which is disposed between the sample chamber 2910 and an exterior space outside of the concentrator 2900 (e.g., atmosphere). In addition, the hydrophobic membrane 2916 is disposed between the volumes of the solution 2914 and the exterior space. The hydrophobic membrane 2916 is permeable to air. In various cases, the hydrophobic membrane 2916 is impermeable to the fluid sample 2912 and the solution 2914. The hydrophobic membrane 2916, for instance, includes PTFE.

During operation, the concentrator 2900 removes at least a portion of at least one solvent from the fluid sample 2914, thereby concentrating a target in the fluid sample 2912. In particular, the solution 2914 includes a first concentration of at least one first solute, the fluid sample 2912 includes a second concentration of at least one second solute, where the first concentration is greater than the second concentration. Due to the discrepancy in concentrations between the fluid sample 2912 and the solution 2914, the solvent(s) spontaneously flow from the fluid sample 2912 into the volumes of the solution 2914 through the membranes 2918. As the solvent(s) move out of the sample chamber 2910, air may flow from the exterior space into the sample chamber 2910 through the hydrophobic membrane 2916. Accordingly, a pressure within the sample chamber 2910 may be maintained at a level equivalent to a pressure of the outer environment during operation of the concentrator 2900. Further, as the solvent(s) move into the solution 2914, the hydrophobic membrane 2916 may release air from a chamber in which the solution 2914 is disposed, which can also prevent a pressure associated with the solution 2914 from increasing above the pressure of the outer environment.

### EXAMPLE CLAUSES

1. A system for detecting an analyte in a urine sample, the system including: a concentrator including: a container; a solution disposed in the container including at least one polymer at a concentration that is greater than a concentration of at least one solute in the urine sample, the at least one solute including the analyte; a membrane disposed inside of the container, the solution being disposed between the membrane and a sidewall of the container, wherein diameters of pores extending through the membrane are shorter than a diameter of the analyte and shorter than a diameter of the at least one polymer; a sample chamber disposed in the container and configured to receive the urine sample, the membrane being disposed between the sample chamber and the solution, the solution being configured to generate a concentrated urine sample by passively removing water from the urine sample through the membrane when the urine sample is disposed in the sample chamber; a hydrophobic membrane disposed between the sample chamber and an outer environment, the hydrophobic membrane being permeable to air and impermeable to the urine sample; and a diagnostic assay including: a porous substrate configured to move the concentrated urine sample via capillary action; a first antibody that specifically binds to the analyte, the first antibody being bound to a tag that emits a detection signal; and a second antibody that specifically binds to the analyte, the second antibody being bound to the porous substrate.

2. The system of clause 1, wherein the at least one solute in the solution includes: polyethylene glycol (PEG) 1500 at a concentration of 2.0 grams per milliliter (g/mL).

3. The system of clause 1 or 2, wherein the solution is configured to generate the concentrated urine sample in 10 to 60 minutes after the urine sample is initially disposed in the sample chamber, and wherein the concentration of the analyte in the concentrated urine sample is at least 100 times greater than the concentration of the analyte in the urine sample.

4. A concentrator, including: a container; a solution disposed in the container including at least one first solute at a concentration that is greater than a concentration of at least one second solute in a fluid sample, the fluid sample including an analyte; and a membrane disposed inside of the container, the solution being disposed between the membrane and a sidewall of the container, diameters of pores extending through the membrane being shorter than a diameter of the analyte and shorter than a diameter of the at least one first solute.

5. The concentrator of clause 4, wherein the sidewall of the container is cylindrical.

6. The concentrator of clause 4 or 5, wherein the solution includes at least one of water or a hydrophilic solvent.

7. The concentrator of any one of clauses 4 to 6, wherein the concentration of the at least one first solute in the solution is greater than or equal to 0.2 grams per milliliter (g/mL).

8. The concentrator of any one of clauses 4 to 7, wherein the concentration of the at least one first solute in the solution is less than or equal to 3 g/mL.

9. The concentrator of any one of clauses 4 to 8, wherein a molecular weight of the at least one first solute is in a range of 1,500 to 1,000,000 Daltons (Da).

10. The concentrator of any one of clauses 4 to 9, wherein the at least one first solute includes at least one of a polymer, a detergent, a surfactant, or a micelle.

11. The concentrator of any one of clauses 4 to 10, wherein the at least one first solute includes at least one of polyethylene glycol (PEG), polystyrene sulfonate (PSS), polyacrylic acid (PAA), polyethyleneimine (PEI), pectin, or sodium dodecyl sulfate (SDS).

12. The concentrator of any one of clauses 4 to 11, wherein the analyte includes at least one of tuberculosis antigen lipoarabinomannan (LAM), human immunodeficiency virus (HIV), human growth hormone (hGH), cell-free DNA, human chorionic gonadotropin (hCG), SARS-CoV-2 nucleocapsid (N) protein, an exosome, ribonucleic acid (RNA), a polysaccharide, a bacterium, or a virus.

13. The concentrator of any one of clauses 4 to 12, wherein the analyte includes human chorionic gonadotropin (hCG) or SARS-CoV-2 N protein.

14. The concentrator of any one of clauses 4 to 13, wherein the fluid sample includes at least one of urine, saliva, or a nasopharyngeal swab.

15. The concentrator of any one of clauses 4 to 14, wherein the diameters of the pores are within a range of 2 nanometers (nm) to 100 nm.

16. The concentrator of any one of clauses 4 to 15, wherein the membrane includes cellulose.

17. The concentrator of any one of clauses 4 to 16, wherein the membrane is a right prism.

18. The concentrator of clause 17, wherein a cross-section of the right prism includes at least one of a circle, a concave polygon, or a convex polygon.

19. The concentrator of any one of clauses 4 to 18, further including: an insert disposed in the container and spaced apart from the membrane by a distance, the membrane being disposed between the insert and the solution.

20. The concentrator of clause 19, wherein the distance is in a range of 0.5 millimeters (mm) to 20 mm.

21. The concentrator of clause 19, wherein a ratio between a surface area of the membrane and a volume of a space disposed between the insert and the membrane is in a range of 1 to 10 centimeters squared per milliliter (cm²/mL).

22. The concentrator of any one of clauses 4 to 21, further including: a collection cap removably coupled to the container and defining a collection volume that is fluidly coupled to a space disposed inside of the container, the membrane being disposed between the solution and the space disposed inside of the container.

23. The concentrator of any one of clauses 4 to 22, further including: a funnel configured to direct the fluid sample into a space disposed inside of the container, the membrane being disposed between the space and a sidewall of the container.

24. The concentrator of any one of clauses 4 to 23, wherein the membrane is disposed between a sample chamber and the solution and the solution is disposed in a solution chamber that is disposed between the membrane and the sidewall of the container, and wherein the concentrator further includes a hydrophobic membrane disposed between the sample chamber and an exterior space outside of the concentrator and disposed between the solution chamber and the exterior space, the hydrophobic membrane being permeable to air and impermeable to the fluid sample.

25. A system including: the concentrator of any one of clauses 4 to 24; and a diagnostic assay configured to detect the presence of the analyte in a sample, a lower detection limit of the diagnostic assay being greater than the concentration of the analyte in the fluid sample.

26. The system of clause 25, wherein the diagnostic assay includes a lateral flow assay.

27. A method performed by a concentrator, the method including: receiving a fluid sample; concentrating an analyte in the fluid sample by extracting water from the fluid sample through a membrane and into a solution including at least one first solute at a concentration that is greater than a concentration of at least one second solute in the fluid sample, diameters of pores extending through the membrane being shorter than a diameter of an analyte in the fluid sample, shorter than a diameter of the at least one first solute, and shorter than a diameter of the at least one second solute; and based on concentrating the analyte in the fluid sample, outputting the fluid sample.

28. The method of clause 27, wherein the concentration of the at least one first solute in the solution is greater than or equal to 0.2 grams per milliliter (g/mL).

29. The method of clause 27 or 28, wherein the concentration of the at least one first solute in the solution is less than or equal to 3 g/mL.

30. The method of any one of clauses 27 to 29, wherein a molecular weight of the at least one first solute is in a range of 1,500 to 1,000,000 Daltons (Da).

31. The method of any one of clauses 27 to 30, wherein the at least one first solute includes at least one of polyethylene glycol (PEG), polystyrene sulfonate (PSS), polyacrylic acid (PAA), polyethyleneimine (PEI), pectin, or sodium dodecyl sulfate (SDS).

32. The method of any one of clauses 27 to 31, wherein the analyte includes at least one of tuberculosis antigen lipoarabinomannan (LAM), human immunodeficiency virus (HIV), human growth hormone (hGH), cell-free DNA, human chorionic gonadotropin (hCG), SARS-CoV-2 nucleocapsid (N) protein, an exosome, ribonucleic acid (RNA), a polysaccharide, a bacterium, or a virus.

33. The method of any one of clauses 27 to 32, wherein a ratio between a surface area of the membrane and the volume of the fluid sample is in a range of 1 to 10 centimeters squared per milliliter (cm²/mL).

34. The method of any one of clauses 27 to 33, wherein the water is passively extracted from the fluid sample.

35. The method of any one of clauses 27 to 34, wherein concentrating the analyte in the fluid sample includes increasing the concentration of the analyte in the fluid sample by 10 to 1,000 times within 10 to 60 minutes.

36. The method of any one of clauses 27 to 35, wherein outputting the fluid sample includes: outputting the fluid sample into a collection cap.

37. The method of any one of clauses 27 to 36, further including: receiving an insert in a sample chamber, a distance between the insert and the sample chamber being in a range of 0.5 millimeters (mm) to 20 mm wherein the fluid sample is disposed inside of the sample chamber and between the insert and the membrane.

38. The method of any one of clauses 27 to 37, wherein receiving the fluid sample includes funneling the fluid sample into a sample chamber, the membrane being disposed between the sample chamber and a sidewall of the container.

39. The method of any one of clauses 27 to 38, wherein the fluid sample includes at least one of blood, urine, blood, saliva, serum, semen, mucus, or a nasopharyngeal swab.

40. The method of clause 39, based on outputting the fluid sample, outputting, by a diagnostic assay, a detection signal indicative of the analyte in the fluid sample by: receiving a sample of the fluid sample at a porous substrate; moving the sample into a detection region of the porous substrate; binding a first antibody to the analyte, the first antibody being conjugated with a tag; and binding a second antibody to the analyte, the second antibody being bound to the porous substrate, wherein the tag outputs the detection signal.

41. A concentrator, including: a membrane, diameters of pores extending through the membrane being shorter than a diameter of a target in a fluid sample; and a solution disposed on a side of the membrane, the solution including at least one first solute at a concentration that is greater than a concentration of at least one second solute in the fluid sample, a diameter of the at least one first solute being greater than the diameters of the pores extending through the membrane.

42. The concentrator of clause 41, wherein the target includes at least one of a biopharmaceutical, a biologic, or an analyte.

43. The concentrator of clause 41 or 42, wherein the concentrator includes a sidewall at least partially enclosing a space, the sidewall including the membrane, the solution being disposed in the space.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (lie), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, lie, Phe, Met, and Trp; Group 11 (aliphatic): Gly, Ala, Val, Leu, and lie; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.](1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M⁻¹, up to 10⁶ M⁻¹, up to 10⁵ M⁻¹. Alternatively, affinity may be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (K off) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A system for detecting an analyte in a urine sample, the system comprising:
a concentrator comprising:
a container;
a solution disposed in the container comprising at least one polymer at a concentration that is greater than a concentration of at least one solute in the urine sample, the at least one solute comprising the analyte;
a membrane disposed inside of the container, the solution being disposed between the membrane and a sidewall of the container, wherein diameters of pores extending through the membrane are shorter than a diameter of the analyte and shorter than a diameter of the at least one polymer;
a sample chamber disposed in the container and configured to receive the urine sample, the membrane being disposed between the sample chamber and the solution, the solution being configured to generate a concentrated urine sample by passively removing water from the urine sample through the membrane when the urine sample is disposed in the sample chamber;
a hydrophobic membrane disposed between the sample chamber and an outer environment, the hydrophobic membrane being permeable to air and impermeable to the urine sample; and
a diagnostic assay comprising:
a porous substrate configured to move the concentrated urine sample via capillary action;
a first antibody that specifically binds to the analyte, the first antibody being bound to a tag that emits a detection signal; and
a second antibody that specifically binds to the analyte, the second antibody being bound to the porous substrate.

2. The system of claim 1, wherein the at least one solute in the solution comprises: polyethylene glycol (PEG) 1500 at a concentration of 2.0 grams per milliliter (g/mL).

3. The system of claim 1, wherein the solution is configured to generate the concentrated urine sample in 10 to 60 minutes after the urine sample is initially disposed in the sample chamber, and
wherein the concentration of the analyte in the concentrated urine sample is at least 100 times greater than the concentration of the analyte in the urine sample.

4. A concentrator, comprising:
a container;
a solution disposed in the container comprising at least one first solute at a concentration that is greater than a concentration of at least one second solute in a fluid sample, the fluid sample comprising an analyte; and
a membrane disposed inside of the container, the solution being disposed between the membrane and a sidewall of the container, diameters of pores extending through the membrane being shorter than a diameter of the analyte and shorter than a diameter of the at least one first solute.

5. The concentrator of claim 4, wherein the sidewall of the container is cylindrical.

6. The concentrator of claim 4, wherein the solution comprises at least one of water or a hydrophilic solvent.

7. The concentrator of claim 4, wherein the concentration of the at least one first solute in the solution is greater than or equal to 0.2 grams per milliliter (g/mL).

8. The concentrator of claim 4, wherein the concentration of the at least one first solute in the solution is less than or equal to 3 g/mL.

9. The concentrator of claim 4, wherein a molecular weight of the at least one first solute is in a range of 1,500 to 1,000,000 Daltons (Da).

10. The concentrator of claim 4, wherein the at least one first solute comprises at least one of a polymer, a detergent, a surfactant, or a micelle.

11. The concentrator of claim 4, wherein the at least one first solute comprises at least one of polyethylene glycol (PEG), polystyrene sulfonate (PSS), polyacrylic acid (PAA), polyethyleneimine (PEI), pectin, or sodium dodecyl sulfate (SDS).

12. The concentrator of claim 4, wherein the analyte comprises at least one of tuberculosis antigen lipoarabinomannan (LAM), human immunodeficiency virus (HIV), human growth hormone (hGH), cell-free DNA, human chorionic gonadotropin (hCG), SARS-CoV-2 nucleocapsid (N) protein, an exosome, ribonucleic acid (RNA), a polysaccharide, a bacterium, or a virus.

13. The concentrator of claim 4, wherein the analyte comprises human chorionic gonadotropin (hCG) or SARS-CoV-2 N protein.

14. The concentrator of claim 4, wherein the fluid sample comprises at least one of urine, saliva, or a nasopharyngeal swab.

15. The concentrator of claim 4, wherein the diameters of the pores are within a range of 2 nanometers (nm) to 100 nm.

16. The concentrator of claim 4, wherein the membrane comprises cellulose.

17. The concentrator of claim 4, wherein the membrane is a right prism.

18. The concentrator of claim 17, wherein a cross-section of the right prism comprises at least one of a circle, a concave polygon, or a convex polygon.

19. The concentrator of claim 4, further comprising:
an insert disposed in the container and spaced apart from the membrane by a distance, the membrane being disposed between the insert and the solution.

20. The concentrator of claim 19, wherein the distance is in a range of 0.5 millimeters (mm) to 20 mm.

21. The concentrator of claim 19, wherein a ratio between a surface area of the membrane and a volume of a space disposed between the insert and the membrane is in a range of 1 to 10 centimeters squared per milliliter (cm²/mL).

22. The concentrator of claim 4, further comprising:
a collection cap removably coupled to the container and defining a collection volume that is fluidly coupled to a space disposed inside of the container, the membrane being disposed between the solution and the space disposed inside of the container.

23. The concentrator of claim 4, further comprising:
a funnel configured to direct the fluid sample into a space disposed inside of the container, the membrane being disposed between the space and a sidewall of the container.

24. The concentrator of claim 4, wherein the membrane is disposed between a sample chamber and the solution and the solution is disposed in a solution chamber that is disposed between the membrane and the sidewall of the container, and
wherein the concentrator further comprises a hydrophobic membrane disposed between the sample chamber and an exterior space outside of the concentrator and disposed between the solution chamber and the exterior space, the hydrophobic membrane being permeable to air and impermeable to the fluid sample.

25. A system comprising:
the concentrator of claim 4; and
a diagnostic assay configured to detect the presence of the analyte in a sample, a lower detection limit of the diagnostic assay being greater than the concentration of the analyte in the fluid sample.

26. The system of claim 25, wherein the diagnostic assay comprises a lateral flow assay.

27. A method performed by a concentrator, the method comprising:
receiving a fluid sample;
concentrating an analyte in the fluid sample by extracting water from the fluid sample through a membrane and into a solution comprising at least one first solute at a concentration that is greater than a concentration of at least one second solute in the fluid sample, diameters of pores extending through the membrane being shorter than a diameter of an analyte in the fluid sample, shorter than a diameter of the at least one first solute, and shorter than a diameter of the at least one second solute; and
based on concentrating the analyte in the fluid sample, outputting the fluid sample.

28. The method of claim 27, wherein the concentration of the at least one first solute in the solution is greater than or equal to 0.2 grams per milliliter (g/mL).

29. The method of claim 27, wherein the concentration of the at least one first solute in the solution is less than or equal to 3 g/mL.

30. The method of claim 27, wherein a molecular weight of the at least one first solute is in a range of 1,500 to 1,000,000 Daltons (Da).

31. The method of claim 27, wherein the at least one first solute comprises at least one of polyethylene glycol (PEG), polystyrene sulfonate (PSS), polyacrylic acid (PAA), polyethyleneimine (PEI), pectin, or sodium dodecyl sulfate (SDS).

32. The method of claim 27, wherein the analyte comprises at least one of tuberculosis antigen lipoarabinomannan (LAM), human immunodeficiency virus (HIV), human growth hormone (hGH), cell-free DNA, human chorionic gonadotropin (hCG), SARS-CoV-2 nucleocapsid (N) protein, an exosome, ribonucleic acid (RNA), a polysaccharide, a bacterium, or a virus.

33. The method of claim 27, wherein a ratio between a surface area of the membrane and the volume of the fluid sample is in a range of 1 to 10 centimeters squared per milliliter (cm²/mL).

34. The method of claim 27, wherein the water is passively extracted from the fluid sample.

35. The method of claim 27, wherein concentrating the analyte in the fluid sample comprises increasing the concentration of the analyte in the fluid sample by 10 to 1,000 times within 10 to 60 minutes.

36. The method of claim 27, wherein outputting the fluid sample comprises:
outputting the fluid sample into a collection cap.

37. The method of claim 27, further comprising:
receiving an insert in a sample chamber, a distance between the insert and the sample chamber being in a range of 0.5 millimeters (mm) to 20 mm
wherein the fluid sample is disposed inside of the sample chamber and between the insert and the membrane.

38. The method of claim 27, wherein receiving the fluid sample comprises funneling the fluid sample into a sample chamber, the membrane being disposed between the sample chamber and a sidewall of the container.

39. The method of claim 27, wherein the fluid sample comprises at least one of blood, urine, blood, saliva, serum, semen, mucus, or a nasopharyngeal swab.

40. The method of claim 39, based on outputting the fluid sample, outputting, by a diagnostic assay, a detection signal indicative of the analyte in the fluid sample by:
receiving a sample of the fluid sample at a porous substrate;
moving the sample into a detection region of the porous substrate;
binding a first antibody to the analyte, the first antibody being conjugated with a tag; and
binding a second antibody to the analyte, the second antibody being bound to the porous substrate,
wherein the tag outputs the detection signal.

41. A concentrator, comprising:
a membrane, diameters of pores extending through the membrane being shorter than a diameter of a target in a fluid sample; and
a solution disposed on a side of the membrane, the solution comprising at least one first solute at a concentration that is greater than a concentration of at least one second solute in the fluid sample, a diameter of the at least one first solute being greater than the diameters of the pores extending through the membrane.

42. The concentrator of claim 41, wherein the target comprises at least one of a biopharmaceutical, a biologic, or an analyte.

43. The concentrator of claim 41, wherein the concentrator comprises a sidewall at least partially enclosing a space, the sidewall comprising the membrane, the solution being disposed in the space.
